(19) United States
(12) Patent Application Publication
(10) Pub. No.: US 2024/0124901 A1
(43) Pub. Date: Apr. 18, 2024

(54) CONTROL SYSTEM, APPRATUS, AND METHOD FOR ENZYME DOSING USING PRIMARY AMINO NITROGEN MEASUREMENT

(71) Applicant: Novozymes A/S, Bagsvaerd (DK)

(72) Inventors: Kyle Rothanzl, Franklinton, NC (US); Patrick E. Williams, Kongens Lyngby (DK); Gregory Michael Ames, Copenhagen SV (DK); Scott Dickerson, Chapel Hill, NC (US); James Waterman, Aimes, IA (US); Laurie Duval, Ottawa (CA); Scott Robert McLaughlin, Wake Forest, NC (US); Nate Parrish, Raleigh, NC (US); Mike A. Smith, Raleigh, NC (US)

(73) Assignee: Novozymes A/S, Bagsvaerd (DK)

(21) Appl. No.: 18/391,488

(22) Filed: Dec. 20, 2023

Related U.S. Application Data
(63) Continuation-in-part of application No. 18/569,099, filed on Jan. 1, 0001.
(60) Provisional application No. 63/209,665, filed on Jun. 11, 2021.
(60) Provisional application No. 63/253,458, filed on Oct. 7, 2021.
(60) Provisional application No. 63/270,941, filed on Oct. 22, 2021.
(60) Provisional application No. 63/312,505, filed on Feb. 22, 2022.

(51) Int. Cl. C12P 7/06

(52) U.S. Cl. CPC C12P 7/06

(57) ABSTRACT

A method of producing bioethanol in a bioethanol system and a control system for a bioethanol system is disclosed, the control system comprising a controller comprising one or more processors and an interface, wherein the one or more processors are configured to obtain a primary amino nitrogen (PAN) measurement; determine an input scheme based on the PAN measurement; and control one or more input devices of the bioethanol system according to the input scheme.

### TECHNICAL FIELD

The present disclosure relates to the production of bioethanol and related control systems, apparatuses and methods. In particular, a method of producing bioethanol in a bioethanol system is disclosed. Embodiments of the present disclosure generally relate to controlling primary amino nitrogen (PAN) levels and/or protease dosing in a bioethanol system, and more specifically to controlling PAN levels, for example, by obtaining a PAN measurement and dosing a protease enzyme based on the PAN measurement to achieve target PAN levels in the bioethanol system. In particular, embodiments of the present disclosure relate to control systems for obtaining a PAN measurement during the production of bioethanol and determining an input scheme based on the PAN measurement, and related methods and apparatuses.

### BACKGROUND

Yeast and enzymes are ingredients used to produce products, such as bioethanol, which may be used to replace fossil fuels. Bioethanol may reduce CO2 emissions while allowing systems that conventionally operate on fossil fuels to continue operating with minimal alterations.

WIPO Pat. Application No. WO2022261162A1 discloses a computer-implemented method for producing bioethanol in a bioethanol system. The method comprises obtaining a grain flour flow of grain flour; determining an input scheme based on the grain flour flow; and controlling one or more input devices of the bioethanol system according to the input scheme. The method allows optimization of slurry preparation and input, by controlling e.g., grain flour dosing, yeast dosing, enzyme dosing, to a tank (e.g., slurry, liquefaction, fermentation, etc.) of a bioethanol plant or system which in turn may lead to increased or optimized bioethanol yield. In particular, the real-time monitoring and determination of the grain flour flow allows more precise control of the preparation process and/or liquefaction and/or fermentation. Using one or more spectrometer sensors (e.g., in-line NIR) to measure the crude nutrient content of the incoming feedstock (percent starch, percent fat, percent protein, percent fiber, and percent water), and by coupling this flour composition data from the NIR with flour mass data from the weight meter (e.g., Ronan device) enables calculating the mass of each component (starch/fat/protein/fiber) going into the process. This will in turn allow more accurate dosing of enzymes into the process, e.g, alpha-amylase dosing based on the mass of starch, protease dosing based on mass of protein, xylanase dosing based on the mass of fiber etc.

Controlling the amount of yeast and/or enzyme used in a process of producing bioethanol effectively may improve the fermentation process, reduce the fermentation time, and/or reduce the amount of yeast and enzymes used. For example, proteases dosed in liquefaction and/or fermentation break down proteins present in the feedstock. Protein hydrolysis before fermentation produces amino acids which serve as a nitrogen source for yeast metabolism. Finished oil yields have also been shown to improve when protease is dosed in liquefaction. However, overdosing of protease can lead to excessive breakdown of protein and affect distillers dried grains (DDG)/distillers dried grains with solubles (DDGS) yields. And underdosing of protease can fail to produce the optimal amount of amino acids needed to maximize yeast performance.

### BRIEF SUMMARY

Accordingly, there is a need for methods and devices for optimizing bioethanol yield and processes related to bioethanol production. The present invention provides a solution to the above problem by provisioning a sensor or sensor device (e.g., in-line mid-infrared (MIR) device to reliably measure primary amino nitrogen (“PAN”) in a bioethanol system, such as post-liquefaction or pre-fermentation). The in-line placement of the device allows for the establishment of a control loop that can produce a liquefact with targeted and optimal PAN levels, allowing fermentations to eliminate underdosing or overdosing of protease to achieve optimal yeast health without affecting DDG/DDGS quality and deteriorating its market value.

A control system for a bioethanol system or parts of a bioethanol system is disclosed, the control system comprising a controller comprising one or more processors and an interface. The one or more processors are configured to obtain, e.g. via the interface, a primary amino nitrogen (“PAN”) measurement; determine an input scheme based on the PAN measurement; and control, e.g. via the interface, one or more input devices of the bioethanol system according to the input scheme.

Further, a method for producing bioethanol in a bioethanol system is disclosed. The method comprises obtaining a PAN measurement; determining an input scheme based on the PAN measurement; and controlling one or more input devices of the bioethanol system according to the input scheme.

The present disclosure allows optimization of slurry preparation and input, by controlling e.g. protease dosing, based on a PAN measurement, to a tank (e.g. liquefaction, fermentation, etc.) of a bioethanol plant or system which in turn may lead to increased or optimized bioethanol yield.

It is an important advantage of the present disclosure that a more accurate and improved protease enzyme dosing is provided which in turn allows for a more stable bioethanol production and reduces waste. In particular, the real-time monitoring and determination of the PAN measurement allows more precise control of the preparation process and/or liquefaction and/or fermentation e.g., dosing of protease, for example, to achieve target PAN levels optimal for yeast growth and fermentation productivity.

Further, the use of in situ real-time online monitoring and control of the bioethanol system allows for a more stable input to the bioethanol system.

In an embodiment, to determine an input scheme comprises to determine an enzyme dosing scheme. The enzyme dosing scheme may comprise a first enzyme flowrate for a first enzyme, and wherein to control one or more input devices comprises to control a first enzyme input device according to the enzyme dosing scheme, wherein the first enzyme is protease.

In an embodiment, to control an input device comprises to control the first enzyme input device according to the first enzyme flowrate.

In an embodiment, the enzyme dosing scheme comprises a second enzyme flowrate for a second enzyme, and wherein to control an input device comprises to control a second enzyme input device according to the second enzyme flowrate. The second enzyme can be selected from alpha-amylase, endoglucanase, glucoamylase, phospholipase, phytase, pullulanase, and xylanase or is an enzyme composition comprising one or more of alpha-amylase, endoglucanase, glucoamylase, phospholipase, phytase, pullulanase, and xylanase.

In an embodiment, to determine an input scheme comprises to determine a feed rate, and wherein to control one or more input devices comprises to control a feeder input device according to the feed rate. In another embodiment, the one or more processes may be configured to obtain a grain flour flow of grain flour. The control system may include a weight meter for provision of weight data of grain flour and wherein to obtain a grain flour flow comprises to determine the grain flour flow based on the weight data.

In an embodiment, the control system comprises a spectrometer for provision of spectrometer data of the PAN measurement. The spectrometer can be situated inside a liquefaction tank of the bioethanol system, in-line with an output of the liquefaction tank, in-line with an input of a fermentation tank of the bioethanol system, inside the fermentation tank, or in-line with an output of the fermentation tank, in-line with an input of a production tank of the bioethanol system, inside the production tank, or in-line with an output of the production tank, in-line with an input of a preparation tank of the bioethanol system that connects to the output of a backset line, inside the preparation tank, or in-line with an output of the preparation tank, in-line with an input of a separation tank of the bioethanol system, or in-line with an output of the separation tank, in-line with an output of the backset line or inside the backset line, in-line with an input of a slurry mixer.

In an embodiment, to determine the first enzyme flow rate for the protease enzyme is based on the PAN measurement.

In an embodiment, the control system comprises a spectrometer for provision of spectrometer data of grain flour and wherein to obtain a grain flour flow comprises to determine a first component flow of a first component of the grain flour based on the spectrometer data and the grain flour flow. The first component can be protein content.

In an embodiment, to obtain a grain flour flow comprises to determine a second component flow of a second component of the grain flour based on the spectrometer data and the grain flour flow. The second component may be one of a starch, a fiber, a fat and a moisture content.

### BRIEF DESCRIPTION OF THE DRAWINGS

While the specification concludes with claims particularly pointing out and distinctly claiming embodiments of the present disclosure, the advantages of embodiments of the disclosure may be more readily ascertained from the following description of embodiments of the disclosure when read in conjunction with the accompanying drawings in which:

FIG. 1 illustrates a schematic view of a bioethanol system in accordance with one or more embodiments of the present disclosure;

FIG. 2 illustrates a block diagram of a bioethanol system in accordance with one or more embodiments of the present disclosure;

FIG. 3 illustrates a block diagram of a yeast injection system in accordance with one or more embodiments of the present disclosure;

FIG. 4 illustrates a block diagram of a control system in accordance with one or more embodiments of the present disclosure;

FIG. 5 illustrates another block diagram of another control system, according to various embodiments of the present disclosure;

FIG. 6 illustrates another block diagram of yet another control system, according to various embodiments of the present disclosure; and

FIG. 7 is a flow chart of an exemplary method of producing bioethanol.

FIG. 8 shows corn mass measured in an ethanol plant by both the rotary feeder and by an embodiment of the control system of the present disclosure in which a weight meter (e.g., Ronan) is used for accurate, real-time measurement of corn mass, illustrating the error of the rotary feeder measurement, which error was predominant in the industry before the control system of the present disclosure.

FIG. 9 illustrates the relationship between the dose of protease enzyme as a % weight of enzyme per weight of ground corn according to the weight meter and the pan_offset is the reading of primary amino nitrogen (“PAN”) from the spectrometer device. There is approximately 180 minutes from when corn is ground and enzyme added until that liquefied mash comes in contact with the spectrometer.

FIG. 10 illustrates the relationship between PAN reading from the spectrometer and ethanol yield. The ethanol yield of the total process is expressed in gallons of absolute ethanol per 56 pounds of corn. It reflects that increasing the PAN increases the ethanol yield.

FIG. 11 illustrates the effect of protease dosing on a PAN measurement from a spectrometer device with respect to time. The red line is the protease dose as a % weight of protease enzyme per weight of corn ground. The blue line is the PAN reading from a spectrometer device. The graph highlights that the protease dose increased by −25% for 1 day and as a result, PAN stayed high for several days afterward due to the water recycling nature of the ethanol process. Excess PAN that is created initially by the protease goes through fermentation, is used by the yeast, and whatever is not used by the yeast recycles back to the cook portion of the process through backset (the water left over after distillation).

### DETAILED DESCRIPTION

The illustrations presented herein are not meant to be actual views of any particular apparatus or system or component thereof, but are merely idealized representations employed to describe illustrative embodiments. The drawings are not necessarily to scale.

As used herein, the term “substantially” in reference to a given parameter means and includes to a degree that one skilled in the art would understand that the given parameter, property, or condition is met with a small degree of variance, such as within acceptable manufacturing tolerances. For example, a parameter that is substantially met may be at least about 90% met, at least about 95% met, at least about 99% met, or even at least about 100% met.

As used herein, relational terms, such as “first,” “second,” “top,” “bottom,” etc., are generally used for clarity and convenience in understanding the disclosure and accompanying drawings and do not connote or depend on any specific preference, orientation, or order, except where the context clearly indicates otherwise.

As used herein, the term “and/or” means and includes any and all combinations of one or more of the associated listed items.

As used herein, the terms “vertical” and “lateral” refer to the orientations as depicted in the figures.

As used herein, the term primary amino nitrogen (“PAN”) refers to the combined concentration of free amino acids, di peptides and/or tri peptides in a bioethanol system processing fluid.

### Control System for Bioethanol System or Parts Thereof

A control system for a bioethanol system or parts of a bioethanol system is disclosed. The control system comprises a controller comprising one or more processors and an interface.

The control system optionally comprises a sensor system. The sensor system comprises one or more sensors connected to controller(s) of the control system for provision of sensor data to the controller. The control system may be a distributed control system. In other words, the control system may comprise a plurality of controllers, each controller implementing one or more control schemes to control the bioethanol system or parts thereof.

The one or more processors of the controller are configured to obtain, such as one or more of determine, measure, receive, and retrieve, PAN and/or a PAN flow. The PAN and/or the PAN flow may be obtained based on sensor data from one or more sensors arranged in the bioethanol system, such as at an outlet of a liquefaction tank of the bioethanol system. In other words, the PAN flow may be obtained as a directly measured mass flow rate of PAN.

The one or more processors of the controller are configured to obtain, such as one or more of determine, measure, receive, and retrieve, a grain flour flow of grain flour and/or a grain flow, e.g. at a conveyor of the bioethanol system. The grain flour flow also denoted GFF and/or the grain flow GF may be obtained based on grain flour data/sensor data from one or more sensors arranged in the bioethanol system, such as at a conveyer or an output of a milling device/grinder. In other words, the grain flour flow and/or the grain flow may be obtained as a directly measured mass flow rate of grain flour.

### Determining a Preparation/Liquefaction Section Input Scheme

The one or more processors of the controller are configured to determine an input scheme based on the PAN measurement, the grain flour flow, or the PAN measurement and the grain flour flow. The input scheme may comprise and define control parameters for one or more input devices in the bioethanol system, e.g. for one or more pump devices, one or more grinders, one or more conveyors or other device(s) operating as input devices in the bioethanol system.

The one or more processors of the controller are configured to control one or more input devices of the bioethanol system according to the input scheme. For example, to control one or more input devices of the bioethanol system may comprise to output or transmit, e.g. via the interface, one or more control parameters also denoted input control parameters ICPs to input device(s) of the bioethanol system.

Enzyme Dosing Scheme

The input scheme may comprise an enzyme dosing scheme. The enzyme dosing scheme is indicative of and defines enzyme control parameters also denoted ECP's for controlling enzyme dosing in the bioethanol system.

In one or more example controllers, to determine an input scheme comprises to determine an enzyme dosing scheme, the enzyme dosing scheme comprising a first enzyme flowrate or first enzyme amount (first enzyme control parameter) for a first enzyme, and wherein to control one or more input devices comprises to control a first enzyme input device according to the enzyme dosing scheme, such as a first enzyme flowrate or first enzyme amount. In other words, the enzyme dosing scheme may comprise a first enzyme control parameter ECP_1, such as a first enzyme flowrate and/or a first enzyme amount or indicative thereof.

The first enzyme may be a first enzyme or a first enzyme composition. In one or more example controllers, the first enzyme is a protease.

In one or more example controllers, to control an input device comprises to control the first enzyme input device according to the first enzyme control parameter, such as first enzyme flowrate and/or the first enzyme amount. In other words, the input scheme may comprise an enzyme dosing scheme comprising a first enzyme control parameter, and the controller may be configured to output or transmit a first enzyme control parameter ECP_1 indicative of or being the first enzyme flowrate and/or the first enzyme amount, e.g. to the first enzyme input device, such as a dosing pump or valve.

In one or more example controllers, the enzyme dosing scheme comprises a second enzyme flowrate or second enzyme amount (second enzyme control parameter) for a second enzyme, and wherein to control an input device comprises to control a second enzyme input device according to the enzyme dosing scheme, such as the second enzyme flowrate and/or second enzyme amount. In other words, the enzyme dosing scheme may comprise a second enzyme control parameter ECP_2, such as a second enzyme flowrate and/or a second enzyme amount or indicative thereof.

The second enzyme may be a second enzyme or a second enzyme composition. The second enzyme composition may comprise the first enzyme and/or one or more additional enzymes.

In one or more example controllers, the second enzyme is selected from alpha-amylase, endoglucanase, glucoamylase, phospholipase, phytase, pullulanase, and xylanase or is a second enzyme composition comprising one or more of alpha-amylase, endoglucanase, glucoamylase, phospholipase, phytase, protease, pullulanase, and xylanase. The second enzyme may be different from the first enzyme. In other words, the controller may be configured to determine and control input of a plurality of enzymes or enzyme compositions, thereby allowing tailoring the input on enzymes to the input of grain flour and/or PAN measurement.

In one or more example controllers, to control an input device comprises to control the second enzyme input device according to the second enzyme control parameter, such as the second enzyme flowrate and/or the second enzyme amount. In other words, the input scheme may comprise an enzyme dosing scheme comprising a second enzyme control parameter, and the controller may be configured to output or transmit a second enzyme control parameter ECP_2 indicative of or being the second enzyme flowrate and/or the second enzyme amount, e.g. to the second enzyme input device, such as a dosing pump or valve.

The second enzyme may operate on a by-product of the first enzymatic process carried out by the first enzyme.

The enzyme dosing scheme may define a rate or a difference between different enzyme flowrates. For example, the enzyme dosing scheme may comprise a first ratio indicative of ratio between a first enzyme flowrate and a second enzyme flowrate. In other words, to control an input device may comprise to control a ratio between input of the first enzyme and input of the second enzyme.

In one or more example controllers, the enzyme dosing scheme comprises a third enzyme flowrate or third enzyme amount (third enzyme control parameter) for a third enzyme, and wherein to control an input device comprises to control a third enzyme input device according to the enzyme dosing scheme, such as the third enzyme flowrate and/or third enzyme amount. In other words, the enzyme dosing scheme may comprise a third enzyme control parameter ECP_3, such as a third enzyme flowrate and/or a third enzyme amount or indicative thereof.

The third enzyme may be a third enzyme or a third enzyme composition. The third enzyme composition may comprise the first enzyme, the second enzyme, and/or one or more additional enzymes.

In one or more example controllers, the third enzyme is selected from alpha-amylase, endoglucanase, glucoamylase, phospholipase, phytase, pullulanase, and xylanase or is a third enzyme composition comprising one or more of alpha-amylase, endoglucanase, glucoamylase, phospholipase, phytase, protease, pullulanase, and xylanase. The third enzyme may be different from the first enzyme and/or different from the second enzyme.

In one or more example controllers, to control an input device comprises to control the third enzyme input device according to the third enzyme control parameter, such as the third enzyme flowrate and/or the third enzyme amount. In other words, the input scheme may comprise an enzyme dosing scheme comprising a third enzyme control parameter, and the controller may be configured to output or transmit a third enzyme control parameter ECP_3 indicative of or being the third enzyme flowrate and/or the third enzyme amount, e.g. to the third enzyme input device, such as a dosing pump or valve.

The third enzyme may operate on a by-product of one or both of the first enzymatic process carried out by the first enzyme and the second enzymatic process carried out by the second enzyme.

In one or more example controllers, the enzyme dosing scheme may comprise a ratio indicative of ratio between a first enzyme flowrate and a third enzyme flowrate. In other words, to control an input device may comprise to control a ratio between input of the first enzyme and input of the third enzyme.

In one or more example controllers, the enzyme dosing scheme may comprise a ratio indicative of ratio between a second enzyme flowrate and a third enzyme flowrate. In other words, to control an input device may comprise to control a ratio between input of the second enzyme and input of the third enzyme.

In one or more example controllers, the enzyme dosing scheme may comprise a ratio indicative of ratio between a combined enzyme flowrate, e.g. flowrate of first enzyme and second enzyme, and a third enzyme flowrate. In other words, to control an input device may comprise to control a ratio between input of the combination of first enzyme/second enzyme and input of the third enzyme.

In one or more example controllers, the enzyme dosing scheme comprises a fourth enzyme flowrate or fourth enzyme amount (fourth enzyme control parameter) for a fourth enzyme, and wherein to control an input device comprises to control a fourth enzyme input device according to the enzyme dosing scheme, such as the fourth enzyme flowrate and/or fourth enzyme amount. In other words, the enzyme dosing scheme may comprise a fourth enzyme control parameter ECP_4, such as a fourth enzyme flowrate and/or a fourth enzyme amount or indicative thereof.

The fourth enzyme may be a fourth enzyme or a fourth enzyme composition. The fourth enzyme composition may comprise the first enzyme, the second enzyme, the third enzyme, and/or one or more additional enzymes.

In one or more example controllers, the fourth enzyme is selected from alpha-amylase, endoglucanase, glucoamylase, phospholipase, phytase, pullulanase, and xylanase or is a fourth enzyme composition comprising one or more of alpha-amylase, endoglucanase, glucoamylase, phospholipase, phytase, protease, pullulanase, and xylanase. The fourth enzyme may be different from the first enzyme and/or different from the second enzyme and/or different from the third enzyme.

The fourth enzyme may operate on a by-product of one or more of the first enzymatic process carried out by the first enzyme, the second enzymatic process carried out by the second enzyme, and/or the third enzymatic process carried out by the third enzyme.

In one or more example controllers, to control an input device comprises to control the fourth enzyme input device according to the fourth enzyme control parameter, such as the fourth enzyme flowrate and/or the fourth enzyme amount. In other words, the input scheme may comprise an enzyme dosing scheme comprising a fourth enzyme control parameter, and the controller may be configured to output or transmit a fourth enzyme control parameter ECP_4 indicative of or being the fourth enzyme flowrate and/or the fourth enzyme amount, e.g. to the fourth enzyme input device, such as a dosing pump or valve.

In other words, to determine an input scheme/enzyme dosing scheme may comprise determining one or more enzyme control parameters optionally including one or more of first enzyme control parameter, second enzyme control parameter, third enzyme control parameter, and fourth enzyme control parameter based on the PAN measurement and/or the grain flour flow.

Feed Rate and/or Feed Scheme

In one or more example controllers, to determine an input scheme comprises to determine a feed rate and/or a feed scheme, e.g. of grain, and wherein to control one or more input devices optionally comprises to control a feeder input device according to the feed rate/feed scheme. The feed scheme optionally comprises a first feed rate or first feed amount for a first feeder input device, e.g. for feeding grain or other raw material to the bioethanol system. To control one or more input devices optionally comprises to control a first feeder input device according to the feed rate/feed scheme, such as a first feed rate or first feed amount. In other words, the feed scheme may comprise a first feed control parameter FCP_1, such as a first feed rate and/or a first feed amount or indicative thereof.

In one or more example controllers, to control an input device comprises to control the first feeder input device according to the first feed control parameter, such as (first) feed rate and/or the (first) feed amount. In other words, the input scheme may comprise a feed rate and/or a feed scheme comprising a first feed control parameter, and the controller may be configured to output or transmit a first feed control parameter FCP_1 indicative of or being the first feed rate and/or the first feed amount, e.g. to the first feeder input device. The first feeder input device may be a rotary valve.

In one or more example controllers, to determine a feed scheme comprises to determine a second feed rate or second feed amount for a second feeder input device, e.g. for feeding grain or other raw material to the bioethanol system, and wherein to control one or more input devices optionally comprises to control a second feeder input device according to the feed scheme, such as the second feed rate or second feed amount for a second feeder input device, e.g. for feeding grain or other raw material to the bioethanol system. In other words, the feed scheme may comprise a second feed control parameter FCP_2, such as a second feed rate and/or a second feed amount or indicative thereof.

In one or more example controllers, to control an input device comprises to control the second feeder input device according to the second feed control parameter, such as second feed rate and/or the second feed amount. In other words, the input scheme may comprise a feed rate and/or a feed scheme comprising a second feed control parameter, and the controller may be configured to output or transmit a second feed control parameter FCP_2 indicative of or being the second feed rate and/or the second feed amount, e.g. to the second feeder input device. The second feeder input device may be a rotary valve.

In one or more example controllers, to determine a feed scheme comprises to determine a third feed rate or third feed amount for a third feeder input device, e.g. for feeding grain or other raw material to the bioethanol system, and wherein to control one or more input devices optionally comprises to control a third feeder input device according to the feed scheme, such as the third feed rate or third feed amount for a third feeder input device, e.g. for feeding grain or other raw material to the bioethanol system. In other words, the feed scheme may comprise a third feed control parameter FCP_3, such as a third feed rate and/or a third feed amount or indicative thereof.

In one or more example controllers, to control an input device comprises to control the third feeder input device according to the third feed control parameter, such as third feed rate and/or the third feed amount. In other words, the input scheme may comprise a feed rate and/or a feed scheme comprising a third feed control parameter, and the controller may be configured to output or transmit a third feed control parameter FCP_3 indicative of or being the third feed rate and/or the third feed amount, e.g. to the third feeder input device. The third feeder input device may be a rotary valve.

### Sensors or Sensor Devices

The control system may comprise one or more sensors including a first sensor or first sensor device, a second sensor or sensor device, a third sensor, or sensor device, a fourth sensor or sensor device, a fifth sensor, or sensor device, and/or a sixth sensor or sensor device. The sensor(s) provide sensor data for the controller(s), e.g. for determining the input scheme based on the sensor data.

First Sensor or Sensor Device

In one or more example control systems, the control system comprises a weight meter (first sensor or first sensor device) for provision of weight data of grain flour and wherein to obtain a grain flour flow comprises to determine the grain flour flow based on the weight data. The weight meter may be a RONAN density meter.

Second Sensor or Sensor Device

In one or more example control systems, the control system comprises a speedometer (second sensor or second sensor device) for provision of speed data, e.g. of a conveyor such as a belt conveyor, and wherein to obtain a grain flour flow comprises to determine the grain flour flow based on the speed data.

Third Sensor or Sensor Device

In a typical single stream, dry-grind ethanol plant using only corn as feedstock, weight meters (e.g., Ronan device) alone can be used to make reasonably accurate enzyme dosing decisions. This, however, assumes that the starch content of the incoming corn is not highly variable and so the mass of corn going into the process is proportional to the mass of flour going into the process. Often this is not the case and particularly in cases where plants are using mixed feedstocks going into the mill. In such cases in order to accurately determine the optimal enzyme dosing scheme (and which classes of enzymes to dose) a more accurate in-line determination of the amounts of starch, protein, fiber, fat, and water is needed.

Therefore, in one or more example control systems, the control system comprises a spectrometer (third sensor or third sensor device) for provision of spectrometer data of grain flour and wherein to obtain a grain flour flow comprises to determine a first component flow or a first component amount of a first component of the grain flour based on the spectrometer data and the grain flour flow. The first component is protein. Accordingly, the first component flow may be a protein flow or amount of protein. In one or more example controllers, the first enzyme is protease and the first component is protein.

The third sensor/sensor device, such as the spectrometer, may be arranged at a (belt) conveyor and/or at an output of a grinder e.g. of a preparation section of the bioethanol system. The spectrometer may comprise one or more infrared sensors, such as near infrared (NIR) sensors, mid-infrared (MIR) sensors, or far-infrared (FIR) sensors. The NIR spectrometer may be a Zeiss NIR spectrometer. The NIR spectrometer may be a BUCHI NIR spectrometer. The NIR spectrometer may be a PERTEN NIR spectrometer. The NIR spectrometer may be a METROHM NIR spectrometer. The spectrometer may be an FTIR spectrometer. Using one or more spectrometer sensors (e.g., in-line NIR) to measure the crude nutrient content of the incoming feedstock (% starch, % fat, % protein, % fiber, and % water), and by coupling this flour composition data from the NIR with flour mass data from the weight meter (e.g., Ronan device) enables calculating the mass of each component (starch/fat/protein/fiber) going into the process. This will in turn allow more accurate dosing of enzymes into the process, e.g, alpha-amylase dosing based on the mass of starch, protease dosing based on mass of protein, xylanase dosing based on the mass of fiber etc. This NIR technology has particular value in the case of mixed feedstocks, where there may be a high degree of variation of the above component contents. The addition of an in-line NIR to the flour feed allows the measurement of e.g., incoming starch regardless of which feedstock it was derived from, or variability in the mixing ratio of multiple feedstocks.

The feedstock may in one embodiment be selected from the group comprising corn, wheat, barley, rye, milo, sago, cassava, tapioca, sorghum, oat, rice, peas, beans, beats, sweet potatoes, other sources of starch, such as e.g., starch waste by-products, or mixtures thereof. In one embodiment the mixed feedstock may comprise corn and milo. In another embodiment the mixed feedstock may comprise corn and wheat.

In one or more example controllers, to determine a first enzyme flowrate or a first enzyme amount for a first enzyme is based on the first component flow. In one or more example controllers, the first enzyme is protease and the first component is protein.

In one or more example controllers, to determine a second enzyme flowrate or a second enzyme amount for a second enzyme is based on the second component flow. In one or more example controllers, the second enzyme is alpha-amylase and the second component is starch.

In one or more example controllers, to determine a third enzyme flowrate or a third enzyme amount for a third enzyme is based on the third component flow. In one or more example controllers, the third enzyme is a xylanase and the third component is fiber.

In one or more example controllers, to determine a fourth enzyme flowrate or a fourth enzyme amount for a fourth enzyme is based on the fourth component flow. In one or more example controllers, the fourth enzyme is lipase (e.g., phospholipase) and the fourth component is fat.

In one or more example controllers, to obtain a grain flour flow comprises to determine a second component flow or a second component amount of a second component of the grain flour based on the spectrometer data and the grain flour flow. The second component may be one of a starch, a fiber, a fat, or a moisture content. Accordingly, the second component flow may be a starch flow, a fiber flow, a fat flow, or a moisture content flow. In one or more example controllers, the second enzyme is alpha-amylase and the second component is starch. In one or more example controllers, the first enzyme is protease and the first component is protein and the second enzyme is alpha-amylase and the second component is starch.

In one or more example controllers, to obtain a grain flour flow comprises to determine a third component flow or a third component amount of a third component of the grain flour based on the spectrometer data and the grain flour flow. The third component may be fiber. Accordingly, the third component flow may be a fiber flow. In one or more example controllers, the third enzyme is xylanase and the third component is fiber. The fiber may comprise cellulose or hemicellulose. In one or more example controllers, the first enzyme is protease and the first component is protein, the second enzyme is alpha-amylase and the second component is starch, and the third enzyme is xylanase and the third component is fiber.

In one or more example controllers, to obtain a grain flour flow comprises to determine a fourth component flow or a fourth component amount of the grain flour based on the spectrometer data and the grain flour flow. The fourth component may be fat. Accordingly, the fourth component flow may be a fat flow. In one or more example controllers, the fourth enzyme is lipase (e.g., phospholipase) and the fourth component is fat. In one or more example controllers, the first enzyme is protease and the first component is protein, the second enzyme is alpha-amylase and the second component is starch, the third enzyme is xylanase and the third component is fiber, and the fourth enzyme is lipase and the fourth component is fat.

A component flow may be a combined component flow for a plurality of components. For example, a component flow, such as the first component flow and/or the second component flow and/or the third component flow and/or the fourth component flow may be a combined component flow, e.g. a component flow of starch and protein, a component flow of starch and fiber, a component flow of protein and fiber, a component flow of starch and fat, a component flow of protein and fat, a component flow of fiber and fat, a component flow of starch, protein and fiber, a component flow of starch, protein and fat, a component flow of protein, fiber and fat, a component flow of starch, fiber and fat, or a component flow of protein, fiber, starch and fat.

In one or more example controllers, to determine a first enzyme flowrate for a first enzyme is based on the first component flow and/or one or more other components flows, such as the second component flow and/or the third component flow of the third component and/or the fourth component flow of the fourth component. In one or more example controllers, to determine a first enzyme flowrate for a first enzyme is based on the first component flow of the first component and/or one or more other component flows, such as the second component flow of the second component, and/or the third component flow of the third component and/or the fourth component flow of the fourth component, and/or of the PAN measurement or PAN flow.

In one or more example controllers, to determine a second enzyme flowrate for a second enzyme is based on the second component flow and/or one or more other component flows, such as the first component flow and/or the third component flow of a third component and/or the fourth component flow of the fourth component. In one or more example controllers, to determine a second enzyme flowrate for a second enzyme is based on the second component flow of the second component and/or one or more other component flows, such as the first component flow of the first component, and/or the third component flow of the third component and/or the fourth component flow of the fourth component, and/or of the PAN measurement or PAN flow.

In one or more example controllers, to determine a third enzyme flowrate for a third enzyme is based on the third component flow and/or one or more other component flows, such as the first component flow of the first component and/or the second component flow of a second component and/or the fourth component flow of the fourth component. In one or more example controllers, to determine a third enzyme flowrate for a third enzyme is based on the third component flow of the third component and/or one or more other component flows, such as the first component flow of the first component, and/or the second component flow of the second component and/or the fourth component flow of the fourth component, and/or of the PAN measurement or PAN flow.

In one or more example controllers, to determine a fourth enzyme flowrate for a fourth enzyme is based on the fourth component flow and/or one or more other component flows, such as the first component flow of the first component and/or the second component flow of a second component and/or the third component flow of the third component. In one or more example controllers, to determine a fourth enzyme flowrate for a fourth enzyme is based on the fourth component flow of the fourth component and/or one or more other component flows, such as the first component flow of the first component, and/or the second component flow of the second component and/or the third component flow of the third component, and/or of the PAN measurement or PAN flow.

Fourth Sensor or Sensor Device

In one or more example control systems, the control system comprises a spectrometer (fourth sensor or fourth sensor device) for provision of spectrometer data of a liquefact/liquefied mash. The fourth sensor/sensor device, such as the spectrometer, may be arranged at near an output of the liquefaction tank of the liquefaction section of the bioethanol system, inside a liquefaction tank of the bioethanol system, in-line with an output of the liquefaction tank, in-line with an input of a fermentation tank of the bioethanol system, inside the fermentation tank, or in-line with an output of the fermentation tank. Positioning of the fourth sensor/sensor device in this way enables the control system to determine the effectiveness of any of the first, second, third, fourth enzymes on the first, second, third, and fourth component flows. That is, the control system can measure, via the fourth sensor/fourth sensor device, the hydrolysis products of the first, second, third, and fourth enzymes acting on the first, second, third and/or fourth components, as well as the remaining component amount or flows of the first, second, third, and/or fourth components. These measurements can be used to further adjust in real-time the first, second, third and/or fourth enzyme flow rates in order to optimize the hydrolysis of the first, second, third and/or fourth components to optimize nutrients for yeast (e.g., glucose and PAN) and improve ethanol yields during fermentation. The spectrometer (fourth sensor or fourth sensor device) can also be used to measure other components of the liquefact/liquefied mash, such as PAN.

In one or more example control systems, to obtain a grain flour flow comprises to determine the first component flow or the first component amount of the first component of the grain flour based on the spectrometer data from the third sensor or third sensor device and/or fourth sensor or fourth sensor device and the grain flour flow.

In one or more example controllers, the spectrometer data of the liquefact/liquefied mash provided by the fourth sensor/sensor device can be used to determine the effectiveness of the first enzyme on the first component flow or the first component amount of the grain flour, and to determine whether the first enzyme flow rate needs to be optimized based on changes in gain flour flow and/or the liquefact/liquefied mash. For example, if the first enzyme is protease and the first component flow is protein, the fourth sensor/sensor device can detect protein (e.g., soluble protein) that can be used by the control system to determine whether adjustments in protease dosing need to be made based on the amount of soluble protein present post-liquefaction as detected by the fourth sensor or fourth sensor device relative to the amount of protein present in the grain flour flow measured by the third sensor or third sensor device.

In one or more example control systems, the fourth sensor/sensor device is configured to obtain a PAN measurement. In one or more example controllers, the spectrometer data of the liquefact/liquefied mash provided by the fourth sensor/sensor device can be used to determine the effectiveness of the first enzyme on the first component flow or the first component amount of the grain flour, and to determine whether the first enzyme flow rate needs to be optimized based on the amount of PAN present in the liquefact/liquefied mash. For example, if the first enzyme is protease and the first component flow is protein, the fourth sensor/sensor device can detect PAN that can be used by the control system to determine whether adjustments in protease dosing need to be made based on the amount of PAN present post-liquefaction as detected by the fourth sensor or fourth sensor device. The skilled artisan will appreciate that the PAN measurement can be used independently of, or together in combination with, the protein measured by the third sensor/sensor device and/or fourth sensor/sensor device to adjust the protease flow rate and optimize protease dosing, for example to achieve target PAN levels optimal for yeast growth and ethanol yields during fermentation.

In one or more example controllers, the spectrometer data of the liquefact/liquefied mash provided by the fourth sensor/sensor device can be used to determine the effectiveness of the second enzyme on the second component flow or the second component amount of the second component of the grain flour, and to determine whether the second enzyme flow rate needs to be optimized based on changes in grain flour flow. For example, if the second enzyme is alpha-amylase and the second component flow is starch, the fourth sensor/sensor device can detect sugar profiles (e.g., DP1, DP2, DP3, DP4, etc.) that can be used by the control system to determine whether adjustments in alpha-amylase dosing need to be made based on the amount of starch remaining post-liquefaction as detected by the fourth sensor or fourth sensor device relative to the amount of starch present in the grain flour flow measured by the third sensor or third sensor device.

In one or more example controllers, the spectrometer data of the liquefact/liquefied mash provided by the fourth sensor/sensor device can be used to determine the effectiveness of the third enzyme on the third component flow or the third component amount of the third component of the grain flour, and to determine whether the third enzyme flow rate needs to be optimized based on changes in grain flour flow. For example, if the third enzyme is xylanase and the third component flow is fiber, the fourth sensor/sensor device can detect fiber and/or fiber hydrolysis products, such as monomeric C5 sugars (e.g., xylose and/or arabinose), that can be used by the control system to determine whether adjustments in xylanase dosing need to be made based on the amount of fiber remaining post-liquefaction as detected by the fourth sensor or fourth sensor device relative to the amount of fiber present in the grain flour flow measured by the third sensor or third sensor device.

In one or more example controllers, the spectrometer data of the liquefact/liquefied mash provided by the fourth sensor/sensor device can be used to determine the effectiveness of the fourth enzyme on the fourth component flow or the fourth component amount of the fourth component of the grain flour, and to determine whether the fourth enzyme flow rate needs to be optimized based on changes in grain flour flow. For example, if the fourth enzyme is a lipase (e.g., phospholipase) and the third component flow is fiber, the fourth sensor/sensor device can detect fat/oil that can be used by the control system to determine whether adjustments in lipase dosing need to be made based on the amount of fat/oil remaining post-liquefaction as detected by the fourth sensor or fourth sensor device relative to the amount of fiber present in the grain flour flow measured by the third sensor or third sensor device.

In one or more example control systems, to determine an input scheme comprises to determine a fermentation input scheme, and the spectrometer data of the liquefact/liquefied mash provided by the fourth sensor/sensor device can be used to determine the fermentation input scheme. The fermentation input scheme can be used to determine the dosing of enzymes and/or yeast in the fermentation section.

Fermentation Section Input Scheme/Enzyme Dozing Scheme

In one or more example control systems, the fourth sensor/sensor device is configured to obtain measurements of starch, fiber, fat, cellulose, hemicellulose, trehalose, etc. present in the liquefact/liquefied mash post-liquefaction and to determine an input scheme comprises to determine a fermentation enzyme dosing scheme based on those measurements.

In one or more example controllers, to determine an input scheme comprises to determine a fermentation enzyme dosing scheme, the fermentation enzyme dosing scheme comprising a fifth enzyme flowrate or fifth enzyme amount (fifth enzyme control parameter) for a fifth enzyme, and wherein to control one or more input devices comprises to control a fifth enzyme input device according to the enzyme dosing scheme, such as a fifth enzyme flowrate or fifth enzyme amount. In other words, the enzyme dosing scheme may comprise a fifth enzyme control parameter ECP_5, such as a fifth enzyme flowrate and/or a fifth enzyme amount or indicative thereof.

The fifth enzyme may be a fifth enzyme or a fifth enzyme composition. In one or more example controllers, the fifth enzyme is alpha-amylase and the enzyme dosing scheme for the fifth enzyme is based on residual starch detected by the fourth sensor or fourth sensor device and provided as spectrometer data of the liquefact/liquefied mash. In one or more example controllers, the fifth enzyme composition comprises alpha-amylase and at least one enzyme selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CIP), chitinase, coumaric acid esterase, cutinase, cyclodextrin glycosyltransferase, deoxyribonuclease, endoglucanase, esterase, expansin, feruloyl esterase, galactosidase, glucuronidase, glucuronoyl esterase, hemicellulase, hydrolase, invertase, isomerase, laccase, ligase, ligninolytic enzyme, lipase (e.g., phospholipase, triacylglycerol lipase, etc.), lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

In one or more example controllers, to control an input device comprises to control the fifth enzyme input device according to the fifth enzyme control parameter, such as fifth enzyme flowrate and/or the fifth enzyme amount. In other words, the input scheme may comprise a fifth dosing scheme comprising a fifth enzyme control parameter, and the controller may be configured to output or transmit a fifth enzyme control parameter ECP_5 indicative of or being the fifth enzyme flowrate and/or the fifth enzyme amount, e.g. to the fifth enzyme input device, such as a dosing pump or valve.

The fifth enzyme may operate on a by-product or remnant of the enzymatic processes carried out by the first enzyme, second, enzyme, third enzyme, and/or fourth enzyme. For example, alpha-amylases may act on residual starch present post-liquefaction.

In one or more example controllers, the enzyme dosing scheme comprises a sixth enzyme flowrate or sixth enzyme amount (sixth enzyme control parameter) for a sixth enzyme, and wherein to control an input device comprises to control a sixth enzyme input device according to the enzyme dosing scheme, such as the sixth enzyme flowrate and/or sixth enzyme amount. In other words, the enzyme dosing scheme may comprise a sixth enzyme control parameter ECP_6, such as a sixth enzyme flowrate and/or a sixth enzyme amount or indicative thereof.

The sixth enzyme may be a sixth enzyme or a sixth enzyme composition. The sixth enzyme composition may comprise the fifth enzyme and/or one or more additional enzymes.

In one or more example controllers, the sixth enzyme is a glucoamylase and the enzyme dosing scheme for the sixth enzyme is based on dextrins detected by the fourth sensor or fourth sensor device and provided as spectrometer data of the liquefact/liquefied mash. In one or more example controllers, the sixth enzyme composition comprises alpha-amylase and glucoamylase. In one or more example controllers, the sixth enzyme composition comprises alpha-amylase and/or glucoamylase and one or more additional enzymes selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CIP), chitinase, coumaric acid esterase, cutinase, cyclodextrin glycosyltransferase, deoxyribonuclease, endoglucanase, esterase, expansin, feruloyl esterase, galactosidase, glucoamylase, glucuronidase, glucuronoyl esterase, hemicellulase, hydrolase, invertase, isomerase, laccase, ligase, ligninolytic enzyme, lipase (e.g., phospholipase, triacylglyerol lipase, etc.), phosphol, lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

The sixth enzyme may be different from the fifth enzyme. In other words, the controller may be configured to determine and control input of a plurality of enzymes or enzyme compositions, thereby allowing tailoring the input of fermentation enzymes to the composition of the spectrometer data of the liquefact/liquefied mash as detected by the fourth sensor/fourth sensor device.

In one or more example controllers, to control an input device comprises to control the sixth enzyme input device according to the sixth enzyme control parameter, such as the sixth enzyme flowrate and/or the sixth enzyme amount. In other words, the input scheme may comprise an enzyme dosing scheme comprising a sixth enzyme control parameter, and the controller may be configured to output or transmit a sixth enzyme control parameter ECP_6 indicative of or being the sixth enzyme flowrate and/or the sixth enzyme amount, e.g. to the sixth enzyme input device, such as a dosing pump or valve.

The sixth enzyme may operate on a by-product of the enzymatic process carried out by the first, second, third, fourth, and or fifth enzymatic processes carried out by the first, second, third, fourth and/or fifth enzymes. For instance, alpha-amylases hydrolyses starch into dextrins, which are subsequently hydrolyzed by glucoamylase into fermentable sugar (e.g., glucose) for yeast.

The enzyme dosing scheme may define a rate or a difference between different enzyme flowrates. For example, the enzyme dosing scheme may comprise a first ratio indicative of ratio between a fifth enzyme flowrate and a sixth enzyme flowrate. In other words, to control an input device may comprise to control a ratio between input of the fifth enzyme and input of the sixth enzyme.

In one or more example controllers, the enzyme dosing scheme comprises a seventh enzyme flowrate or seventh enzyme amount (seventh enzyme control parameter) for a seventh enzyme, and wherein to control an input device comprises to control a seventh enzyme input device according to the enzyme dosing scheme, such as the seventh enzyme flowrate and/or seventh enzyme amount. In other words, the enzyme dosing scheme may comprise a seventh enzyme control parameter ECP_7, such as a seventh enzyme flowrate and/or a seventh enzyme amount or indicative thereof.

The seventh enzyme may be a seventh enzyme or a seventh enzyme composition. The seventh enzyme composition may comprise the seventh enzyme, the sixth enzyme, the fifth enzyme and/or one or more additional enzymes.

In one or more example controllers, the seventh enzyme is a trehalase and the enzyme dosing scheme for the fifth enzyme is based on trehalose detected by the fourth sensor or fourth sensor device and provided as spectrometer data of the liquefact, liquefied mash. In one or more example controllers, the seventh enzyme composition comprises alpha-amylase, glucoamylase, and trehalase. In one or more example controllers, the seventh enzyme composition comprises alpha-amylase, glucoamylase, trehalase, and one or more additional enzymes selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CIP), chitinase, coumaric acid esterase, cutinase, cyclodextrin glycosyltransferase, deoxyribonuclease, endoglucanase, esterase, expansin, feruloyl esterase, galactosidase, glucoamylase, glucuronidase, glucuronoyl esterase, hemicellulase, hydrolase, invertase, isomerase, laccase, ligase, ligninolytic enzyme, lipase (e.g., phospholipase, triacylglyerol lipase, etc.), phosphol, lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

The seventh enzyme may be different from the fifth enzyme and/or different from the sixth enzyme.

In one or more example controllers, to control an input device comprises to control the seventh enzyme input device according to the seventh enzyme control parameter, such as the seventh enzyme flowrate and/or the seventh enzyme amount. In other words, the input scheme may comprise an enzyme dosing scheme comprising a seventh enzyme control parameter, and the controller may be configured to output or transmit a seventh enzyme control parameter ECP_7 indicative of or being the seventh enzyme flowrate and/or the seventh enzyme amount, e.g. to the seventh enzyme input device, such as a dosing pump or valve.

The seventh enzyme may operate on a by-product of one or more of the enzymatic process carried out by the first, second, third, fourth, fifth, and/or sixth enzymatic processes carried out by the first, second, third, fourth, fifth, and/or sixth enzymes.

In one or more example controllers, the enzyme dosing scheme may comprise a ratio indicative of ratio between a fifth enzyme flowrate and a seventh enzyme flowrate. In other words, to control an input device may comprise to control a ratio between input of the fifth enzyme and input of the seventh enzyme.

In one or more example controllers, the enzyme dosing scheme may comprise a ratio indicative of ratio between a sixth enzyme flowrate and a seventh enzyme flowrate. In other words, to control an input device may comprise to control a ratio between input of the sixth enzyme and input of the seventh enzyme.

In one or more example controllers, the enzyme dosing scheme may comprise a ratio indicative of ratio between a combined enzyme flowrate, e.g. flowrate of fifth enzyme and sixth enzyme, and a seventh enzyme flowrate. In other words, to control an input device may comprise to control a ratio between input of the combination of fifth enzyme/sixth enzyme and input of the seventh enzyme.

In one or more example controllers, the enzyme dosing scheme comprises an eighth enzyme flowrate or eighth enzyme amount (eighth enzyme control parameter) for an eighth enzyme, and wherein to control an input device comprises to control an eighth enzyme input device according to the enzyme dosing scheme, such as the eighth enzyme flowrate and/or eighth enzyme amount. In other words, the enzyme dosing scheme may comprise an eighth enzyme control parameter ECP_8, such as a eighth enzyme flowrate and/or a eighth enzyme amount or indicative thereof.

The eighth enzyme may be an eighth enzyme or an eighth enzyme composition. The eighth enzyme composition may comprise the fifth enzyme, the sixth enzyme, the seventh enzyme, and/or one or more additional enzymes.

In one or more example controllers, the eighth enzyme is a cellulase. In one or more example controllers, the seventh enzyme is part of a composition and the seventh enzyme composition comprises a beta-glucosidase, a cellobiohydrolase and an endoglucanase. In one or more example controllers, the seventh enzyme composition comprises a beta-glucosidase, a cellobiohydrolase, an endoglucanase, and one or more additional enzymes selected from the group consisting of alpha-amylase, glucoamylase, and trehalase. In one or more example controllers, the seventh enzyme composition comprises alpha-amylase, glucoamylase, trehalase, and one or more additional enzymes selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CIP), chitinase, coumaric acid esterase, cutinase, cyclodextrin glycosyltransferase, deoxyribonuclease, endoglucanase, esterase, expansin, feruloyl esterase, galactosidase, glucoamylase, glucuronidase, glucuronoyl esterase, hemicellulase, hydrolase, invertase, isomerase, laccase, ligase, ligninolytic enzyme, lipase (e.g., phospholipase, triacylglyerol lipase, etc.), phosphol, lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

The eighth enzyme may operate on a by-product of one or more of the enzymatic process carried out by the first, second, third, fourth, fifth, sixth, and/or seventh enzymatic processes carried out by the first, second, third, fourth, fifth, sixth and/or seventh enzymes.

In one or more example controllers, to control an input device comprises to control the eighth enzyme input device according to the eighth enzyme control parameter, such as the eighth enzyme flowrate and/or the eighth enzyme amount. In other words, the input scheme may comprise an enzyme dosing scheme comprising a eighth enzyme control parameter, and the controller may be configured to output or transmit a eighth enzyme control parameter ECP_8 indicative of or being the eighth enzyme flowrate and/or the eighth enzyme amount, e.g. to the eighth enzyme input device, such as a dosing pump or valve.

In one or more example controllers, the enzyme dosing scheme comprises a ninth enzyme flowrate or ninth enzyme amount (ninth enzyme control parameter) for a ninth enzyme, and wherein to control an input device comprises to control a ninth enzyme input device according to the enzyme dosing scheme, such as the ninth enzyme flowrate and/or ninth enzyme amount. In other words, the enzyme dosing scheme may comprise a ninth enzyme control parameter ECP_9, such as a ninth enzyme flowrate and/or a ninth enzyme amount or indicative thereof.

The ninth enzyme may be a ninth enzyme or a ninth enzyme composition. The ninth enzyme composition may comprise the fifth enzyme, the sixth enzyme, the seventh enzyme, the eighth enzyme, and/or one or more additional enzymes.

In one or more example controllers, the ninth enzyme is a hemicellulase. In one or more example controllers, the ninth enzyme is part of a composition, and the ninth enzyme composition comprises an arabinofuranose and a xylanase. In one or more example controllers, the ninth enzyme composition comprises an arabinofuranosidase, a xylanase and a beta-xylosidase. In one or more example controllers, the ninth enzyme composition comprises an arabinofuranonsidase, a xylanase, a beta-xylosidase. In one or more example controllers, the ninth composition comprises at least one arabinofuranosidase, a xylanase, a beta-xylosidase and at least one esterase (e.g., acetylxylan esterase and/or feruloyl esterase). The ninth composition may further comprise one or more additional enzymes selected from the group consisting of alpha-amylase, glucoamylase, and trehalase. In one or more example controllers, the seventh enzyme composition comprises alpha-amylase, glucoamylase, trehalase, and one or more additional enzymes selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CIP), chitinase, coumaric acid esterase, cutinase, cyclodextrin glycosyltransferase, deoxyribonuclease, endoglucanase, esterase, expansin, feruloyl esterase, galactosidase, glucoamylase, glucuronidase, glucuronoyl esterase, hemicellulase, hydrolase, invertase, isomerase, laccase, ligase, ligninolytic enzyme, lipase (e.g., phospholipase, triacylglyerol lipase, etc.), phosphol, lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

The ninth enzyme may operate on a by-product of one or more of the enzymatic process carried out by the first, second, third, fourth, fifth, sixth, seventh and/or eighth enzymatic processes carried out by the first, second, third, fourth, fifth, sixth, seventh and/or eighth enzymes.

In one or more example controllers, to control an input device comprises to control the ninth enzyme input device according to the ninth enzyme control parameter, such as the ninth enzyme flowrate and/or the ninth enzyme amount. In other words, the input scheme may comprise an enzyme dosing scheme comprising a ninth enzyme control parameter, and the controller may be configured to output or transmit a ninth enzyme control parameter ECP_9 indicative of or being the ninth enzyme flowrate and/or the ninth enzyme amount, e.g. to the ninth enzyme input device, such as a dosing pump or valve.

In other words, to determine an input scheme/enzyme dosing scheme may comprise determining one or more enzyme control parameters optionally including one or more of fifth enzyme control parameter, sixth enzyme control parameter, seventh enzyme control parameter, eighth enzyme control parameter, or ninth enzyme control parameter based on the spectrometer data of the liquefact/liquefied mash as detected by the fourth sensor or fourth sensor device.

In one or more example controllers, the enzyme dosing scheme comprises a tenth enzyme flowrate or tenth enzyme amount (tenth enzyme control parameter) for a tenth enzyme, and wherein to control an input device comprises to control a tenth enzyme input device according to the enzyme dosing scheme, such as the tenth enzyme flowrate and/or tenth enzyme amount. In other words, the enzyme dosing scheme may comprise a tenth enzyme control parameter ECP_10, such as a tenth enzyme flowrate and/or a tenth enzyme amount or indicative thereof.

The tenth enzyme may be a tenth enzyme or a tenth enzyme composition. The tenth enzyme composition may comprise the fifth enzyme, the sixth enzyme, the seventh enzyme, the eighth enzyme, ninth enzyme, and/or one or more additional enzymes.

In one or more example controllers, the tenth enzyme is a protease or a composition comprising a protease. In one or more example controllers, the tenth enzyme composition comprises a protease and one or more additional enzymes selected from the group consisting of alpha-amylase, glucoamylase, and trehalase. In one or more example controllers, the tenth enzyme composition comprises alpha-amylase, glucoamylase, trehalase, and one or more additional enzymes selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CIP), chitinase, coumaric acid esterase, cutinase, cyclodextrin glycosyltransferase, deoxyribonuclease, endoglucanase, esterase, expansin, feruloyl esterase, galactosidase, glucoamylase, glucuronidase, glucuronoyl esterase, hemicellulase, hydrolase, invertase, isomerase, laccase, ligase, ligninolytic enzyme, lipase (e.g., phospholipase, triacylglyerol lipase, etc.), phosphol, lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

The tenth enzyme may operate on a by-product of one or more of the enzymatic process carried out by the first, second, third, fourth, fifth, sixth, seventh, eighth and/or ninth enzymatic processes carried out by the first, second, third, fourth, fifth, sixth, seventh, eighth and/or ninth enzymes.

In one or more example controllers, to control an input device comprises to control the tenth enzyme input device according to the tenth enzyme control parameter, such as the tenth enzyme flowrate and/or the tenth enzyme amount. In other words, the input scheme may comprise an enzyme dosing scheme comprising a tenth enzyme control parameter, and the controller may be configured to output or transmit a tenth enzyme control parameter ECP_10 indicative of or being the tenth enzyme flowrate and/or the tenth enzyme amount, e.g. to the tenth enzyme input device, such as a dosing pump or valve.

In one or more example controllers, the enzyme dosing scheme comprises an eleventh enzyme flowrate or eleventh enzyme amount (eleventh enzyme control parameter) for a eleventh enzyme, and wherein to control an input device comprises to control a eleventh enzyme input device according to the enzyme dosing scheme, such as the eleventh enzyme flowrate and/or eleventh enzyme amount. In other words, the enzyme dosing scheme may comprise an eleventh enzyme control parameter ECP_11, such as an eleventh enzyme flowrate and/or an eleventh enzyme amount or indicative thereof.

The eleventh enzyme may be an eleventh enzyme or an eleventh enzyme composition. The eleventh enzyme composition may comprise the fifth enzyme, the sixth enzyme, the seventh enzyme, the eighth enzyme, ninth enzyme, tenth enzyme and/or one or more additional enzymes.

In one or more example controllers, the eleventh enzyme is a phytase or a composition comprising a phytase. In one or more example controllers, the eleventh enzyme composition comprises a phytase and one or more additional enzymes selected from the group consisting of alpha-amylase, glucoamylase, and trehalase. In one or more example controllers, the seventh enzyme composition comprises phytase, alpha-amylase, glucoamylase, trehalase, and one or more additional enzymes selected from the group consisting of acetylmannan esterase, acetylxylan esterase, alpha-galactosidase, alpha-glucosidase, alpha-amylase, aminopeptidase, amylase, arabinanase, arabinofuranosidase, beta-galactosidase, beta-glucosidase, beta-xylosidase, carbohydrase, carboxypeptidase, catalase, cellobiohydrolase, cellulase, cellulose inducible protein (CI P), chitinase, coumaric acid esterase, cutinase, cyclodextrin glycosyltransferase, deoxyribonuclease, endoglucanase, esterase, expansin, feruloyl esterase, galactosidase, glucoamylase, glucuronidase, glucuronoyl esterase, hemicellulase, hydrolase, invertase, isomerase, laccase, ligase, ligninolytic enzyme, lipase (e.g., phospholipase, triacylglyerol lipase, etc.), phosphol, lyase, lytic polysaccharide monooxygenase (e.g., AA9 polypeptide, AA10 polypeptide, AA11 polypeptide, AA13 polypeptide), mannanase, mannosidase, mutanase, oxidoreductase, oxidase, pectinase or pectinolytic enzyme, peroxidase, phytase, polyphenoloxidase, protease or proteolytic enzyme, ribonuclease, swollenin, transferase, transglutaminase, trehalase, xylanase, or xylosidase.

The eleventh enzyme may operate on a by-product of one or more of the enzymatic process carried out by the first, second, third, fourth, fifth, sixth, seventh, eighth, ninth and/or tenth enzymatic processes carried out by the first, second, third, fourth, fifth, sixth, seventh, eighth, ninth, and/or tenth enzymes.

In one or more example controllers, to control an input device comprises to control the eleventh enzyme input device according to the eleventh enzyme control parameter, such as the eleventh enzyme flowrate and/or the eleventh enzyme amount. In other words, the input scheme may comprise an enzyme dosing scheme comprising an eleventh enzyme control parameter, and the controller may be configured to output or transmit an eleventh enzyme control parameter ECP_11 indicative of or being the eleventh enzyme flowrate and/or the eleventh enzyme amount, e.g. to the eleventh enzyme input device, such as a dosing pump or valve.

In other words, to determine an input scheme/enzyme dosing scheme may comprise determining one or more enzyme control parameters optionally including one or more of fifth enzyme control parameter, sixth enzyme control parameter, seventh enzyme control parameter, eighth enzyme control parameter, ninth enzyme control parameter, tenth enzyme control parameter, or eleventh enzyme control parameter based on the spectrometer data of the liquefact/liquefied mash as detected by the fourth sensor or fourth sensor device.

Yeast Dosing Scheme

In one or more example controllers, to determine an input scheme comprises to determine a yeast dosing scheme, the yeast dosing scheme comprising a first yeast flowrate or a first yeast amount of a first yeast (first yeast control parameter), and wherein to control one or more input devices comprises to control a first yeast input device according to the yeast dosing scheme, such as the first yeast flowrate or first yeast amount. In other words, the yeast dosing scheme may comprise a first yeast control parameter YCP_1, such as a first yeast flowrate and/or a first yeast amount or indicative thereof. The first yeast may be a first yeast or a first yeast composition. The first yeast may be formulated as a cream yeast or a dry yeast. A cream yeast may allow precise yeast dosing in an input device.

In one or more example controllers, the first yeast is selected from Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or is a first yeast composition comprising one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp.

In one or more example controllers, to control an input device comprises to control the first yeast input device according to the first yeast control parameter, such as first yeast flowrate and/or the first yeast amount. In other words, the input scheme may comprise a yeast dosing scheme comprising a first yeast control parameter, and the controller may be configured to output or transmit a first yeast control parameter YCP_1 indicative of or being the first yeast flowrate and/or the first yeast amount, e.g. to the first yeast input device, such as a dosing pump or valve.

In one or more example controllers, the yeast dosing scheme comprises a second yeast flowrate or a second yeast amount of a second yeast (second yeast control parameter), and wherein to control an input device comprises to control a second yeast input device according to the second yeast flowrate.

In one or more example controllers, the yeast dosing scheme comprises a second yeast flowrate or a second yeast amount of a second yeast (second yeast control parameter), and wherein to control one or more input devices comprises to control a second yeast input device according to the yeast dosing scheme, such as the second yeast flowrate or second yeast amount. In other words, the yeast dosing scheme may comprise a second yeast control parameter YCP_2, such as a second yeast flowrate and/or a second yeast amount or indicative thereof. The second yeast may be a second yeast or a second yeast composition. The second yeast may be formulated as a cream yeast or a dry yeast. A cream yeast may allow precise yeast dosing in an input device.

In one or more example controllers, the second yeast is selected from Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or is a first yeast composition comprising one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp.

In one or more example controllers, to control an input device comprises to control the second yeast input device according to the second yeast control parameter, such as second yeast flowrate and/or the second yeast amount. In other words, the input scheme may comprise a yeast dosing scheme comprising a second yeast control parameter, and the controller may be configured to output or transmit a second yeast control parameter YCP_2 indicative of or being the second yeast flowrate and/or the second yeast amount, e.g. to the second yeast input device, such as a dosing pump or valve.

In other words, to determine an input scheme/yeast dosing scheme may comprise determining one or more yeast control parameters optionally including a first yeast control parameter and/or a second yeast control parameter, based on the PAN measurement and/or grain flour flow.

The spectrometer (fourth sensor/fourth sensor device) may comprise one or more infrared sensors, such as near infrared (NIR) sensors, mid-infrared (MIR) sensors, or far-infrared (FIR) sensors. The NIR spectrometer may be a PERTEN NIR spectrometer. The NIR spectrometer may be a METROHM NIR spectrometer. The spectrometer (fourth sensor/fourth sensor device) may comprise one or more mid infrared (MIR) sensors, near infrared (NIR) sensors, or far-infrared (FIR) sensors. The MIR spectrometer may be a KEIT MIR spectrometer. The spectrometer may be an FTIR spectrometer.

Fifth Sensor or Sensor Device

In one or more example control systems, the control system comprises a spectrometer (fifth sensor or fifth sensor device) for provision of spectrometer data of the fermentation section (e.g., liquefied mash/liquefact entering the fermenter, fermenting mash inside the fermentation tank, fermented mash leaving the fermentation tank).

The fifth sensor/sensor device, such as the spectrometer, may be arranged at near an output of the liquefaction tank of the liquefaction section of the bioethanol system, inside a liquefaction tank of the bioethanol system, in-line with an output of the liquefaction tank, or in-line with an input of a fermentation tank of the bioethanol system. Positioning of the fifth sensor/sensor device in this way enables the control system to determine the effectiveness of any of the first, second, third, fourth enzymes on the first, second, third, and fourth component flows. That is, the control system can measure, via the fifth sensor/fifth sensor device independently of, or in combination with, measurements of fourth sensor/fourth sensor device, the hydrolysis products of the first, second, third, and fourth enzymes acting on the first, second, third and/or fourth components, as well as the remaining component amount or flows of the first, second, third, and/or fourth components. These measurements can be used to further adjust in real-time the first, second, third and/or fourth enzyme flow rates in order to optimize the hydrolysis of the first, second, third and/or fourth components to optimize nutrients for yeast (e.g., glucose and PAN) and improve ethanol yields during fermentation. The spectrometer (fifth sensor or fifth sensor device) can also be used to measure other components of the liquefact/liquefied mash, such as PAN.

The fifth sensor/sensor device, such as the spectrometer, may also be arranged inside the fermentation tank, or in-line with an output of the fermentation tank. Positioning of the fifth sensor/sensor device in this way enables the control system to determine the effectiveness of any of the fifth, sixth, seventh, eighth, ninth, tenth, or eleventh enzymes or enzyme compositions on the first, second, third, and fourth component flows or their effectiveness on the products and/or by-products of the enzymatic reactions of the first, second, third, and/or fourth enzymes on the first, second, third and fourth component flows, as well their effectiveness on the products and/or by-products of the fifth, sixth, seventh, eighth, ninth, tenth, or eleventh enzymes or enzyme compositions.

That is, the control system can measure, via the fifth sensor/fifth sensor device, the hydrolysis products of the fifth, sixth, seventh, eighth, ninth, tenth, and/or eleventh enzymes or enzyme compositions acting on all of: (1) the hydrolysis products of the first, second, third and/or fourth enzymes acting on the first, second, third and/or fourth components in the liquefaction section; (2) the remaining flows of the first, second, third and/or fourth components following that hydrolysis; and (3) the hydrolysis products of the fifth, sixth, seventh, eighth, ninth, tenth and/or eleventh enzymes or enzyme compositions.

These measurements can be used to further adjust in real-time the first, second, third and/or fourth enzyme flow rates in order to optimize the hydrolysis of the first, second, third and/or fourth components to optimize nutrients for yeast (e.g., glucose and PAN) and improve ethanol yields during fermentation. The measurements can also be used to further adjust in real-time the fifth, sixth, seventh, eighth, ninth, tenth, and/or eleventh enzyme or enzyme composition flow rates in order to optimize the efficiency of hydrolysis during fermentation to maximize the fermentable sugars available for yeast to consume during fermentation and consequently maximize ethanol yields.

The spectrometer (fifth sensor or fifth sensor device) can also be used to measure other components of the fermenting/fermented mash, such as starch, fiber, fat, protein, and PAN. Measurements of PAN post-fermentation can be compared by the control system to measurements of PAN post-liquefaction to determine how much PAN yeast are consuming during fermentation, which can then be used to further adjust protease dosing during liquefaction and/or fermentation, for example to achieve target PAN levels optimal for a particular yeast strain.

In one or more example controllers, to obtain a grain flour flow comprises to determine the first component flow or the first component amount of the first component of the grain flour based on the spectrometer data from the third sensor or third sensor device, the fourth sensor or fourth sensor device, and/or the fifth sensor or fifth sensor device and the grain flour flow.

In one or more example controllers, the spectrometer data of the fermentation tank input and/or output provided by the fifth sensor/sensor device can be used to determine the effectiveness of the first enzyme on the first component flow or the first component amount of the first component of the grain flour, and to determine whether the first enzyme flow rate needs to be optimized based on changes in the PAN. For example, if the first enzyme is protease and the first component flow is protein, the fifth sensor/sensor device can detect PAN released by the hydrolysis of protein by protease post-liquefication, in the fermentation tank and/or after fermentation. The PAN measured can be used by the control system to determine whether adjustments in protease dosing need to be made based on the amount of PAN measured post-liquefaction, in the fermentation tank and/or after fermentation as detected by the fifth sensor or fifth sensor device.

The spectrometer (fifth sensor/fifth sensor device) may comprise one or more near infrared (NIR) sensors one or more infrared sensors, such as near infrared (NIR) sensors, mid-infrared (MIR) sensors, or far-infrared (FIR) sensors. The NIR spectrometer may be a PERTEN NIR spectrometer. The NIR spectrometer may be a METROHM NIR spectrometer. The spectrometer (fifth sensor/fifth sensor device) may comprise one or more mid infrared (MIR) sensors, near infrared (NIR) sensors, or far-infrared (FIR) sensors. The MIR spectrometer may be a KEIT MIR spectrometer. The spectrometer may be an FTIR spectrometer.

Sixth Sensor or Sensor Device

In one or more example control systems, the control system comprises a spectrometer (sixth sensor or sixth sensor device) for provision of spectrometer data of a backset, for example, to detect an amount of PAN present in backset that is available for recycling to the slurry. The sixth sensor/sensor device, such as the spectrometer, may be arranged at near an output of the backset line of the bioethanol system, inside the backset line of the bioethanol system, in-line with an output of the backset line, in-line with an input of a slurry mixer of the bioethanol system, in-line with an input of a preparation tank of the bioethanol system, inside the preparation tank, or in-line with an output of the preparation tank. Positioning the sixth sensor or sixth sensor device in this way enables the control system to determine an accurate amount of PAN that is going to be recycled to the slurry. The skilled artisan will appreciate, however, that the sixth sensor or sixth sensor device can also be positioned in-line with an input of a production tank of the bioethanol system, inside the production tank, or in-line with an output of the production tank, in-line with an input of a separation tank of the bioethanol system, or in-line with an output of the separation tank.

In one or more example controllers, the spectrometer data of the backset provided by the sixth sensor/sensor device can be used to determine the effectiveness of the first enzyme on the first component flow or the first component amount of the first component of the grain flour, and to determine whether the first enzyme flow rate needs to be optimized based on changes in the PAN. For example, if the first enzyme is protease and the first component flow is protein, the sixth sensor/sensor device can detect the remaining PAN after the fermentation and/or after the production of bioethanol. The PAN measured can be used by the control system to determine whether adjustments in protease dosing need to be made based on the amount of PAN measured post-liquefaction as detected by the fourth sensor or fourth sensor device, the amount of PAN measured post-fermentation as detected by the fifth sensor or fifth sensor device, and/or the amount of PAN measured in the backset line by the sixth sensor.

The spectrometer (sixth sensor/sixth sensor device) may comprise one or more near infrared (NIR) sensors one or more infrared sensors, such as near infrared (NIR) sensors, mid-infrared (MIR) sensors, or far-infrared (FIR) sensors. The NIR spectrometer may be a PERTEN NIR spectrometer. The NIR spectrometer may be a METROHM NIR spectrometer. The spectrometer (sixth sensor/sixth sensor device) may comprise one or more mid infrared (MIR) sensors, one or more near infrared (NIR) sensors, or far-infrared (FIR) sensors. The MIR spectrometer may be a KEIT MIR spectrometer. The spectrometer may be an FTIR spectrometer.

The present disclosure relates to a method of producing bioethanol in a bioethanol system, wherein the method comprises obtaining a PAN measurement; determining an input scheme based on the PAN measurement; and controlling one or more input devices of the bioethanol system according to the input scheme.

The present disclosure relates to a method of producing bioethanol in a bioethanol system, wherein the method comprises obtaining a grain flour flow of grain flour; determining an input scheme based on the grain flour flow; and controlling one or more input devices of the bioethanol system according to the input scheme.

The present disclosure relates to a method of producing bioethanol in a bioethanol system, wherein the method comprises obtaining a PAN measurement and a grain flour flow of grain flour; determining an input scheme based on the PAN measurement and the grain flour flow; and controlling one or more input devices of the bioethanol system according to the input scheme.

It is noted that descriptions and features of controller and controller functionality also applies to methods and vice versa.

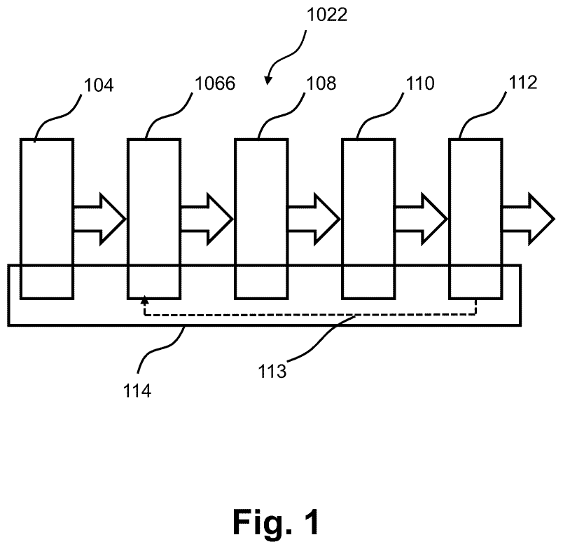

FIG. 1 shows an exemplary bioethanol system or bioethanol plant implementing a control system according to the present disclosure. A bioethanol system 102 comprises a preparation section 104 where grain is milled to flour and combined with water (and optionally enzymes) to form a slurry, a liquefaction section 106 where high temperature is used in combination with enzymes, such as alpha-amylase and/or protease, to hydrolyze the starch present in the slurry to a dextrins and the protease present in the slurry to produce PAN, a fermentation section 108 where the dextrins are saccharified via enzymes, such as glucoamylase, to fermentable sugars and the fermentable sugars and PAN are used by a fermenting organism (e.g., yeast) to produce ethanol, an ethanol production section 110, and a separation section 112. A control system 114 controls devices and operations in one or more of the sections 104, 106, 108, 110, 112. The control system 114 may be a distributed controls system with a central controller and one or more section controllers distributed in respective sections 104, 106, 108, 110, 112.

In a process for producing ethanol, the liquid fermentation products are recovered from the fermented mash e.g., by distillation, which separates the desired fermentation product, e.g. ethanol, from other liquids and/or solids. The remaining fraction is referred to as “whole stillage”, i.e., the fraction left behind after distillation of the fermented mash. The whole stillage is separated into a solid and a liquid fraction, e.g., by centrifugation. The separated solid fraction is referred to as “wet cake” (or “wet grains”) and the separated liquid fraction is referred to as “thin stillage”, i.e., liquid fraction of Stillage after distillation. Wet cake and thin stillage contain about 35 and 7% solids, respectively. Wet cake, with optional additional dewatering in a dryer section, is used as a component in animal feed or is dried to provide “Distillers Dried Grains” (DDG) used as a component in animal feed.

Thin stillage is typically evaporated to provide evaporator condensate and syrup or may alternatively be recycled to the slurry mixer 216 or the preparation section 104 as “backset”. Evaporator condensate or clarified thin stillage may be recycled to the slurry tank or preparation section 104 as “cook water”. The syrup may be blended into DDG or added to the wet cake before or during the drying process, to produce DDGS (Distillers Dried Grain with Solubles). A backset line 113 may connect the thin stillage and/or clarified thin stillage (as backset) from the separation section 112 to the preparation section 104 or to the slurry mixer 216.

The control system 114 for the bioethanol system 102 or respective sections 104, 106, 108, 110, 112 may include a controller including one or more processors and an interface. The control system 114 may include a sensor system. The sensor system may include one or more sensors connected to controller(s) of the control system 114 for provision of sensor data to the controller. The control system 114 may be a distributed control system. In other words, the control system 114 may include a plurality of controllers, each controller implementing one or more control schemes to control the bioethanol system 102, respective sections 104, 106, 108, 110, 112, or parts thereof.

The control system 114 may include one or more sensors. The sensor(s) provide sensor data for the controller(s), which may process the sensor data and perform calculations and/or make determinations based on the sensor data. The control system 114 may include a weight meter, such as a density meter, for measuring weight data of grain flour, slurries, and/or other fluids throughout the bioethanol system 102. For example, a grain flour flow may be determined based on the grain flour flow based on the weight data. The control system 114 may further include a speedometer or flowmeter for measuring speed data, such as the speed of a conveyor or flow velocity. For example, the grain flour flow may be determined based on the speed data.

The control system 114 may include a spectrometer for measuring spectrometer data of grain flour and/or the mixtures within the respective sections 104, 106, 108, 110, 112 of the bioethanol system 102. The spectrometer may be used to determine a component flow, a component amount, an organic acid content, etc., of components of the grain flour and/or the mixtures within the respective sections 104, 106, 108, 110, 112 of the bioethanol system 102 based on the spectrometer data. For example, the components may be one of a starch, a protein, a fiber, a fat, or moisture content. Accordingly, the component flow may be a starch flow or amount of starch, a protein flow or amount of protein, a fiber flow or amount of fiber, a fat flow, or amount of fat, and moisture content or amount of moisture. The spectrometer may also be used to determine a PAN flow or an amount of PAN.

The spectrometer, may be arranged at a (belt) conveyor, at an output of a grinder of the preparation section 104, at an output of the preparation section 104, at an input of the preparation section 104 that connects to the output of the backset line 113, at an output of the liquefaction section 106, at an output of the fermentation section 108, at an input of the fermentation section 108, at an output of the production section 110, at an output of the separation section 112, and/or at an input of the separation section 112. The spectrometer may be a near infrared (NIR) spectrometer, mid-infrared (MIR) spectrometer, far-infrared (FIR) spectrometer or a Fourier transform infrared (FTIR) spectrometer.

The one or more processors of the controller are configured to determine, measure, receive, and retrieve, process conditions, such as a grain flour flow, a grain flow, fermentation time, fermentation solids present, process temperatures, fermentation products, fermentation by-products, such as glycerol and organic acids (e.g., acetic acid, lactic acid, etc.) present, in different locations throughout the bioethanol system 102, based on sensor readings throughout the bioethanol system 102. For example, the one or more processors of the control system 114 may determine process conditions before a mill or grinding element in the preparation section 104, after the mill or grinding element in the preparation section 104, on a conveyor of the bioethanol system 102, entering the liquefaction section 106, within the liquefaction section 106, leaving the liquefaction section 106, entering the fermentation section 108, within the fermentation section 108, leaving the fermentation section 108, entering the production section 110, within the production section 110, leaving the production section 110, entering the separation section 112, within the separation section 112, leaving the separation section 112, etc.

The grain flour flow also denoted GFF and/or the grain flow GF may be obtained based on grain flour data/sensor data from one or more sensors arranged in the bioethanol system, such as at the conveyer or an output of the mill or grinding element in the preparation section 104. In other words, the grain flour flow and/or the grain flow may be obtained as a directly measured mass flow rate of grain flour.

The PAN flow or PAN measurement may be obtained based on PAN data/sensor data from one or more sensors arranged in the bioethanol system, such as within the liquefaction section 106, leaving the liquefaction section 106 (e.g., in line with the output of the liquefication tank), entering the fermentation section 108, within the fermentation section 108, or the leaving the fermentation section 108, entering the slurry mixer 216 through a backset line 113, within the backset line 113, entering the production section 104, leaving the production section 104, entering the separation section 112, leaving the separation section 112, entering the dryer section, leaving the dryer section In other words, the PAN measurement may be obtained as a directly measured concentration and/or mass flow rate of PAN.

The one or more processors of the controller are configured to determine an input scheme based on the PAN measurement and/or the grain flour flow. The input scheme may comprise and define control parameters for one or more input devices in the bioethanol system 102, e.g. for one or more pump devices, one or more grinders, one or more conveyors or other device(s) operating as input devices in the bioethanol system 102.

The one or more processors of the controller are configured to control one or more input devices of the bioethanol system 102 according to the input scheme. For example, controlling one or more input devices of the bioethanol system 102 may include outputting or transmitting one or more control parameters also denoted input control parameters ICPs to the input device(s) of the bioethanol system 102, such as through a direct connection, the interface described above, and/or a network connection between individual controllers.

One or more of the controllers may determine an input scheme to determine an enzyme dosing scheme. The enzyme dosing scheme may control the amount of different enzymes introduced in the liquefaction section 106, in the fermentation section 108, in the preparation section 104, and/or in the separation section 112. The enzyme dosing scheme may include a first enzyme flowrate or first enzyme amount (first enzyme control parameter or ECP_1) for a first enzyme. The one or more controllers may then control a first enzyme input device, such as a dosing pump or dosing valve, according to the enzyme dosing scheme, such as controlling the first enzyme input device to the first enzyme flowrate or first enzyme amount.

The first enzyme may be a first enzyme or a first enzyme composition. For example, the first enzyme is protease, or is a first enzyme composition comprising protease. The first enzyme composition may further comprise one or more of alpha-amylase, endoglucanase, glucoamylase, phospholipase, phytase, protease, pullulanase, or xylanase. The enzyme dosing scheme of the first enzyme may be based on the PAN measurement and/or the protein amount and/or protein flow. The controller may be configured to determine and control input of the first enzyme or enzyme compositions, thereby tailoring the input on the first enzyme to the PAN measurement and/or the protein amount and/or protein flow.

The enzyme dosing scheme may further include a second enzyme flowrate or second enzyme amount (e.g., second enzyme control parameter or ECP_2) for a second enzyme. The one or more controllers may control a second enzyme input device according to the enzyme dosing scheme, such as the second enzyme flowrate and/or second enzyme amount. The second enzyme may be alpha-amylase, endoglucanase, glucoamylase, phospholipase, phytase, pullulanase, and xylanase or is a second enzyme composition comprising one or more of alpha-amylase, endoglucanase, glucoamylase, phospholipase, phytase, pullulanase, and xylanase. The second enzyme may be different from the first enzyme. In other words, the controller may be configured to determine and control input of a plurality of enzymes or enzyme compositions, thereby allowing tailoring the input on enzymes to the input of grain flour. The enzyme dosing scheme may include additional enzyme control parameters, such as third enzyme control parameters (ECP_3), fourth enzyme control parameters (ECP_4), fifth enzyme control parameters (ECP_5), sixth enzyme control parameters (ECP_6), seventh enzyme control parameters (ECP_7), eighth enzyme control parameters (ECP_8), ninth enzyme control parameters (ECP_9), tenth enzyme control parameters (ECP_10), or eleventh enzyme control parameter (ECP_11) for controlling the input of additional enzymes, such as a third enzyme, a fourth enzyme, a fifth enzyme, a sixth enzyme, a seventh enzyme, an eighth enzyme, a ninth enzyme, a tenth enzyme and an eleventh enzyme.

The input scheme may also include a yeast dosing scheme. The yeast dosing scheme may define yeast control parameters for controlling yeast dosing in the bioethanol system 102, such as yeast dosing in the fermentation section 108 of the bioethanol system 102.

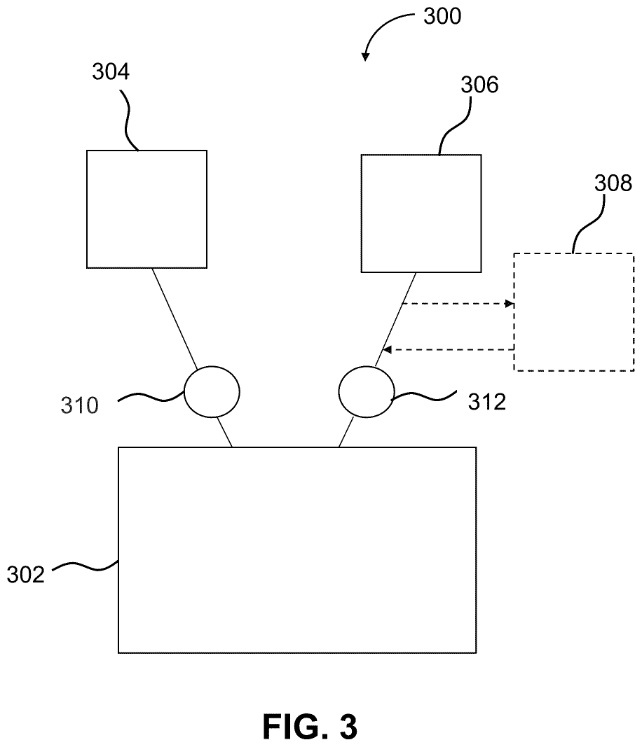

The yeast dosing scheme may include a first yeast flowrate or a first yeast amount of a first yeast (e.g., a first yeast control parameter or YCP_1). The controller may control a first yeast input device (e.g., yeast input device 310 (FIG. 3)), such as a dosing pump or valve, according to the yeast dosing scheme. For example, the first yeast input device may be controlled to the first yeast flowrate or first yeast amount. The first yeast may be formulated as a cream yeast or a dry yeast. A cream yeast may allow precise yeast dosing in an input device. The first yeast may be one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or a composition thereof.

The yeast dosing scheme may include a second yeast flowrate or a second yeast amount of a second yeast (e.g., second yeast control parameter or YCP_2). The controller may control a second yeast input device (e.g., yeast input device 312 (FIG. 3)), such as a dosing pump or valve, according to the second yeast flowrate. The second yeast may be a second yeast or a second yeast composition. The second yeast may be formulated as a cream yeast or a dry yeast. A cream yeast may allow precise yeast dosing in an input device. The second yeast may be one or more of Saccharomyces, Rhodotorula, Schizosaccharomyces, Kluyveromyces, Pichia, Hansenula, Rhodosporidium, Candida, Yarrowia, Lipomyces, Cryptococcus, and Dekkera sp., or a composition thereof.

Determining an input scheme or yeast dosing scheme may include determining one or more yeast control parameters based on the grain flour flow. Determining an input scheme or yeast dosing scheme may include determining one or more yeast control parameters based on the PAN measurement or PAN flow. The yeast dosing scheme may further be determined based on a yeast blend ratio. The yeast blend ratio may be determined based on one or more mathematical, statistical, or machine learning models that predict fermentation product yield (e.g., ethanol) from a controlled fermentation process based on one or more process conditions (also referred to herein as “operating conditions”). The process conditions may be one or more of known or unknown (such as in the case of a machine learning model) indications of fermentation performance. Non-limiting examples of the one or more process conditions include: fermentation time, fermentation solids present, process temperatures, fermentation products, fermentation by-products, such as glycerol and organic acids (e.g., acetic acid, lactic acid) present, etc. Values for the one or more process conditions may be captured by, or determined based on, sensor readings in one or more of the preparation section 104, liquefaction section 106, fermentation section 108, or ethanol production section 110 or lab testing on samples taken from one or more of the preparation section 104, liquefaction section 106, fermentation section 108, or ethanol production section 110. For example, the fermentation time may be estimated using rate tags, such as flour feed rate, beer feed rate, slurry to fermentation rate, etc. The fermentation solids may be estimated using solids measurements from samples in a lab, slurry density measurements, flour mass, etc. The temperature may be determined from temperature readings in the liquefaction section 106, temperature readings the fermentation section 108, ambient temperature readings, and/or temperature differentials in a cooling plant of the fermentation section 108. The fermentation products and fermentation by-products may be estimated using measurements from samples in a lab, such as high-performance liquid chromatography (HPLC), or in-line measurement devices, such as spectrometers (e.g., FTIR spectrometers, MIR spectrometers, and NIR spectrometers).

The yeast blend ratio may be determined based on two or more types of yeast exhibiting at least one unique fermentation characteristic, including but not limited to ability to produce different fermentation products or co-products (including but not limited to lipids, alcohols, proteins, esters, oils, etc.) substrate conversion efficiency (where substrate is C5 or C6 sugars), low by-product formation (glycerol, acetic acid, lactic acid, succinic acid, carbon dioxide, and biomass), robustness (tolerance to one or more of high-temperature and/or high concentrations of organic acids, fusel alcohols, osmotic stress), fermentation kinetics (fast or slow fermenting at the beginning, middle, or end of fermentation), nitrogen utilization efficiency (ability to use wide variety of amino nitrogen sources and/or decreased exogenous sources of urea (urea and/or ammonia)), fermentation time (less than 48, 48 to 54, 54 to 65, or above 65 hours). For example, a yeast blend may include a yeast with high substrate conversion efficiency (Innova Element) paired with a yeast developed for robustness (Innova Force). Non-limiting examples of additional yeast having one or more of the above unique fermentation characteristics include Innova Force, Innova Drive, Innova Element, Innova Fit, Tranform Yield, YP3, CVS, Synerxia, Ruby, Sapphire, Ethanol Red.

The yeast blend may include yeast types capable of metabolizing the same sugars. For example, the high-conversion efficiency yeast and robustness yeast may both be capable of metabolizing C5 sugars. The yeast blend may include yeast types capable of metabolizing different sugars. For example, the high-conversion efficiency yeast capable of metabolizing C5 sugars and the robustness yeast may be capable of metabolizing C6 sugars. Various yeast combinations (e.g., high-conversion efficiency and robustness or C5 and C6 yeasts) are within the scope of this disclosure.

The input scheme may include a feed rate and/or a feed scheme, e.g. of grain. The one or more controllers may control a feeder input device according to the feed rate/feed scheme. The feed scheme may include a first feed rate or first feed amount for a first feeder input device, e.g. for feeding grain or other raw material to the preparation section 104 of the bioethanol system 102. The one or more controllers may control a first feeder input device, such as a rotary valve, according to the feed rate/feed scheme, such as a first feed rate or first feed amount (e.g., a first feed control parameter FCP_1). The feed scheme may further include additional feed control parameters, such as a second feed control parameter (FCP_2), a third feed control parameter (FCP_3), etc., configured to define the control parameters of respective feeder input devices, such as a second feeder input device and a third feeder input device.

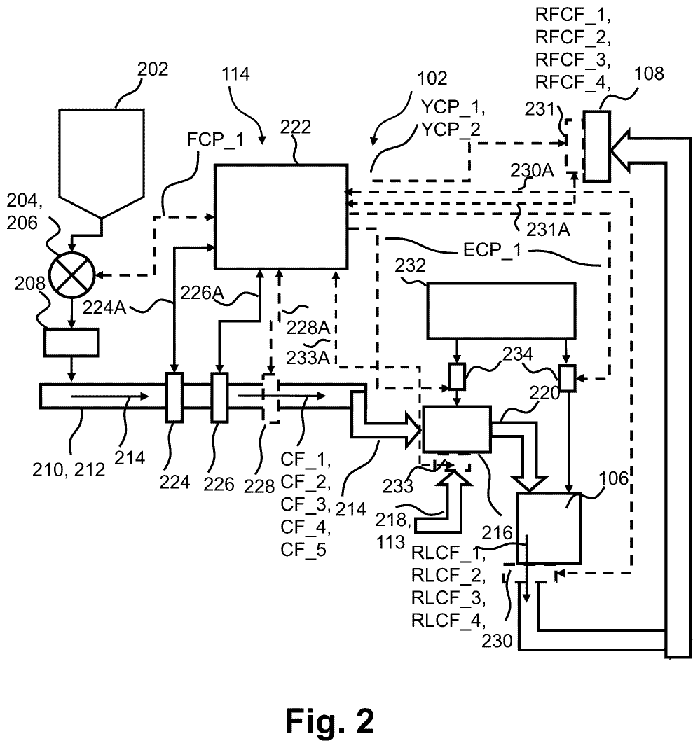

FIG. 2 shows a more detailed block diagram of the bioethanol system 102 according to the present disclosure. The preparation section 104 may include a grain container or grain bin 202 having an output connected to feeder input device 204 including a rotary feeder 206. The feeder input device 204 feeds grain, such as corn, wheat, barley, rye, milo, sago, cassava, tapioca, sorghum, oat, rice, peas, beans, beats, sweet potatoes, or mixtures thereof, to a milling device 208 that may include one or more hammer mills and/or grinders. The milling device outputs grain flour to conveyor 210, such as a conveyor belt 212. The conveyor 210 feeds the grain flour 214 to a mixing device 216, where the grain flour 214 is mixed with liquid 218, such as backset cook water and/or fresh water. The output or slurry 220 from the mixing device 216 may then be fed to liquefaction section 106.

The control system 114 includes a controller 222 including one or more processors and an interface. The control system 114 may include one or more sensors wired or wirelessly connected to the interface of the controller 222. The one or more sensors may include a first sensor 224 and/or a second sensor 226 respectively feeding first sensor data 224A and second sensor data 226A to the controller 222. The one or more sensors may further include a third sensor 228 feeding third sensor data 228A to the controller 222. The one or more sensors may also include a fourth sensor 230 feeding fourth sensor data 230A to the controller 222. The one or more sensors may also include a fifth sensor 231 feeding fifth sensor data 231A to the controller 222. The one or more sensors may also include a sixth sensor 233 feeding sixth sensor data 233A to the controller 222.

The first sensor 224 is arranged at the conveyor 210 and configured to sense or measure one or more properties of the grain flour 214 output from the milling device 208. The first sensor data 224A may include one or more properties, such as a weight, of the grain flour 214 passing the first sensor 224, a speed of the grain flour 214 passing the first sensor 224, and/or spectrometer data of the grain flour 214 passing the first sensor 224.

The second sensor 226 and the third sensor 228 may also be arranged at the conveyor 210 and configured to sense or measure one or more properties of the grain flour 214, such as a weight of the grain flour 214 passing the second sensor 226 and/or the third sensor 228, a speed of the grain flour 214 passing the second sensor 226 and/or the third sensor 228, and/or spectrometer data of the grain flour 214 passing the second sensor 226 and/or the third sensor 228.

The one or more processors of the controller 222 are configured to obtain a grain flour flow of grain flour 214 and/or component flows of (CF_1, CF_2, CF_3, CF_4, CF_5, etc.) by receiving first sensor data 224A, second sensor data 226A, and/or third sensor data 228A.

The fourth sensor 230 and/or fifth sensor 231 may be a spectrometer including one or more NIR, FIR, FTIR, or MIR spectrometers for measuring spectrometer data of a liquefact/liquefied mash as the fourth sensor data 230A and/or for measuring spectrometer data of a fermented mash as the fifth sensor data 231A. The fourth sensor 230 may be situated in the liquefaction tank of liquefaction section 106 to obtain the spectrometer data of the liquefact/liquefied mash as the fourth sensor data 230A. The fourth sensor 230 may be situated in-line with the outlet of the liquefaction tank of liquefaction section 106 to obtain the spectrometer data of the liquefact/liquefied mash as the fourth sensor data 230A. The fourth sensor 230 and/or fifth sensor 231 may be situated in an inlet entering a fermentation tank in the fermentation section 108 to obtain the spectrometer data of the liquefact/liquefied mash as the fourth sensor data 230A and/or fifth sensor data 231A. The fourth sensor 230 and/or fifth sensor 231 may be situated in a fermentation in the fermentation section 108 to obtain the spectrometer data of the fermenting mash in the fermentation section 108. The fourth sensor 230 and/or the fifth sensor 231 may be situated in an outlet leaving the fermentation tank of fermentation section 108 to obtain the spectrometer data of the fermented mash as the fourth sensor data 230A and/or fifth sensor data 231A.

The sixth sensor 233 may be a spectrometer including one or more NIR, FIR, FTIR, or MIR spectrometers for measuring spectrometer data of a liquid 218 (backet) entering into the slurry mixer 216 through the backset lines the sixth sensor data 233A. The backset line 113 may connect the thin stillage and/or clarified thin stillage (as backset) from the separation section 112 to the preparation section 104 or to the slurry mixer 216. The sixth sensor 233 may be situated in the backset line 113 to measure the PAN.

The sixth sensor 233 may be situated in the backset line 113, input or output of the production section 110, input of output of the separation section 112, and/or in-line with an input of a slurry mixer. The sixth sensor 233 may configured to measure PAN, enzymes such as alpha-amylase, protease, xylanase, etc. present in the thin stillage (backset).

The one or more processors of the controller 222 are configured to optimize enzyme dosing in the preparation section 104, in the slurry mixer 216, in the production section 110, and/or in the separation section 112 by receiving the sixth sensor data 233A.

The controller 222 may determine remaining liquefication component flows RLCF_1, RLCF_2, etc., such as a remaining liquefication starch flow, a remaining liquefication protein flow, a remaining liquefication fiber flow, a remaining liquefication fat flow, etc., by comparing component flow CF_x from one of the earlier sensors 224, 226, 228 to the associated component flow measured by the fourth sensor 230. For example, the remaining liquefication first component flow RLCF_1 or remaining liquefication first component flow amount may be determined as the amount of the first component flow CF_1 remaining at the end of liquefaction based on the difference of the first component flow or first component amount determined by the earlier sensor data 224A, 226A, or 228A and the first component flow or first component amount of the fourth sensor data 230A.

The controller 222 may determine remaining fermentation component flows RFCF_1, RFCF_2, etc., such as a remaining fermentation protein flow, a remaining fermentation starch flow (residual starch at the end of fermentation), a remaining fiber and/or a fat/oil at the end of fermentation etc., by comparing the remaining liquefication component flows RLCF_x and/or component flow CF_x from one of the earlier sensors 224, 226, 228, 230 to the associated component flow measured by the fifth sensor 231. For example, the remaining fermentation first component flow RFCF_1 or remaining fermentation first component flow amount may be determined as the amount of the first component flow CF_1 and/or first component liquefication flow RLCF_1 remaining at the end of fermentation determined based on the difference of the first component flow and/or the remaining liquefication first component flow or first component amount determined by the earlier sensor data 224A, 226A, 228A, 230A and the first component flow or first component amount of the fifth sensor data 231A.

In an embodiment, the first component is protein and the remaining liquefication first component flow RLCF_1 or remaining first component flow amount is determined based on the difference of the protein flow or protein amount determined by the earlier sensor data 224A, 226A or 228A and the protein flow or protein component amount determined the fourth sensor data 230A.

In an embodiment, the first component is protein and the remaining fermentation first component flow RFCF_1 or remaining fermentation first component flow amount is determined based on the difference of the protein flow or protein amount determined by the fourth sensor data 230A and/or the fifth sensor data 231A.

[In an embodiment, the first component is protein and the remaining production first component flow RPCF_1 or remaining production first component flow amount is determined based on the difference of the protein flow or protein amount determined by the sixth sensor data 233A.]

It should be appreciated that the first sensor, 224, second sensor 226, third sensor 228, fourth sensor 230, fifth sensor 231 and sixth sensor 233 can be configured to simultaneously determine multiple component flows and/or component amounts in real-time.

The fourth sensor 230 may be configured to determine properties of the liquefied mash 220 exiting the liquefaction section 106 and/or entering the fermentation section 108. For example, the fourth sensor 230 may include one or more of a velocity sensor or flow rate sensor to measure a flow rate of the liquefied mash, a density meter or weight meter to measure a liquefied mash density, a temperature sensor to measure a temperature of the liquefied mash 220, or a spectrometer to determine the PAN contained in the liquefied mash 220.

The one or more processors of the controller 222 are configured to determine an input scheme based on the PAN measurement/flow and/or one or more of the sensor readings from the sensors 224, 226, 228, 230, 231, 233 The input scheme includes one or more of an enzyme dosing scheme, and feed rate/feed scheme.

For example, the fourth sensor 230 may be a spectrometer including one or more NIR or MIR or FIR sensors for measuring spectrometer data of a liquefact/liquefied mash as the fourth sensor data 230A. The fourth sensor 230 may be situated in-line with the output of the liquefaction tank 106 or the fermentation tank 108 and/or inside the liquefaction tank 106 or the fermentation tank 108 in the bioethanol process to obtain the spectrometer data of the liquefact/liquefied mash as the fourth sensor data 230A. To obtain a grain flour flow of grain flour 214 optionally comprises to determine a remaining first component flow RCF_1, such as a remaining protein flow, based on the spectrometer data from one of the other sensors 224, 226, 228, the fourth sensor data/spectrometer data 230A, and the grain flour flow GFF. For example, the remaining first component liquefaction flow RFCF_1 or remaining liquefaction first component flow amount may be determined as a fraction of the first component flow CF_1 remaining at the end of liquefaction determined based on the difference of the first component flow or first component amount determined by the spectrometer sensor data and the first component flow or first component amount of the fourth sensor data 230A. The remaining liquefaction first component flow or first component amount at the end of liquefaction may be used to adjust dosing of the first enzyme or first enzyme flow rate. For example, if the first enzyme is protease and the first component flow is protein, the fourth sensor/sensor device can detect PAN that can be used by the control system to determine whether adjustments in protease dosing need to be made based on the amount of measured PAN post-liquefaction as detected by the fourth sensor or fourth sensor device.

The one or more processors of the controller 222 are configured to determine an input scheme based on the PAN measurement, the grain flour flow GFF and/or one or more component flows, such as first component flow CF_1 of a first component of the grain flour and/or a second component flow CF_2 of a second component of the grain flour and/or a third component flow CF_3 of a third component of the grain flour and/or a fourth component flow CF_4 of a fourth component of the grain flour and/or a fifth component flow CF_5 of a fifth component of the grain flour. The input scheme includes one or more of an enzyme dosing scheme, and feed rate/feed scheme, respectively defining enzyme dosing, and grain feed in the bioethanol system.

The one or more processors of the controller 222 are configured to control one or more input devices of the bioethanol system according to the input scheme. For example, the one or more processors of the controller 222 may be configured to transmit feed control scheme/feed control parameter(s) FCP to the input feeder input device 204. The feeder input device 204 operates accordingly to feed grain from grain bin 202 to milling device 208.

The preparation section 104 and/or the liquefaction section 106 may comprise an enzyme system comprising one or more enzyme containers including first enzyme container 233 and one or more enzyme input devices including a first enzyme input device 234. The one or more processors of the controller 222 may be configured to transmit enzyme dosing scheme/one or more enzyme control parameters including ECP_1 to the first enzyme input device 234 configured to input first enzyme in the first enzyme container 233 to the mixing device 216, thereby adding and mixing first enzyme to the grain flour 214 and liquid 218 according to the enzyme dosing scheme.

The one or more processors of the controller 222 may be configured to determine a yeast dosing scheme, the yeast dosing scheme comprising a first yeast flowrate of a first yeast. The controller 222 may control the input devices comprises according to the yeast dosing scheme by transmitting a first yeast control parameter YCP_1 and/or a second yeast control parameter YCP_2 to a yeast system 300.

FIG. 3 illustrates the yeast system 300. The yeast system 300 may include a first yeast container 304 and a second yeast container 306. The yeast containers 304 and 306 may be coupled to a mixing chamber 302 through respective yeast input devices 310, 312. The mixing chamber 302 may be a part of the fermentation section 108 where the yeast from the yeast system 300 mixes with the liquefied mash 220 to begin the fermentation process. In some embodiments, the mixing chamber 302 may be separate from the fermentation section 108, such that a yeast blend is injected into the fermentation section 108 from the mixing chamber 302.

As described above, the yeast input devices 310, 312 may be controlled based on the yeast dosing scheme. The yeast dosing scheme may include both a total amount or total flow rate of yeast and a yeast blend ratio, which may determine the ratio of a first yeast from the first yeast container 304 to be input through the first yeast input device 310 and a second yeast from the second yeast container 306 to be input through the second yeast input device 312.

In some embodiments, one or more of the first yeast and the second yeast may be a dry yeast. The dry yeast may be re-hydrated in a rehydration chamber 308 before being injected through the respective yeast input device 312, 310. For example, as illustrated in FIG. 3, if the second yeast is a dry yeast, the dry yeast may pass into the rehydration chamber 308 after leaving the second yeast container 306 and before reaching the second yeast input device 312. In the rehydration chamber 308 the dry second yeast may be rehydrated into a cream yeast. Once the dry second yeast is rehydrated, the second yeast may pass through the second yeast input device 312 as a cream yeast, which may be more precisely controlled than the dry yeast.

The yeast system 300 may be maintained at a temperature lower than room temperature (e.g., less than about 20° C.). For example, the yeast system 300 may be refrigerated. Maintaining the yeast system 300 at a temperature below room temperature may maintain the yeast in a substantially stable condition. Maintaining the yeast in a substantially stable condition may allow the yeast to reserve the energy to be released during the fermentation process in the fermentation section 108 rather than releasing the energy in the yeast system 300 prior to being inserted into the fermentation section 108.

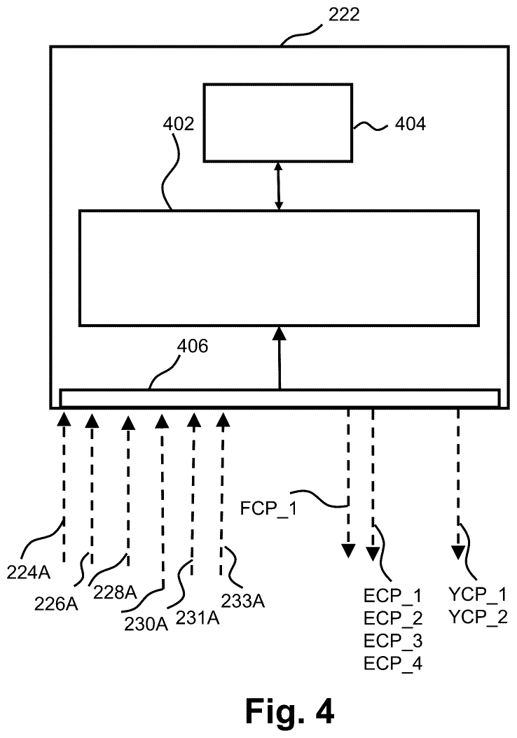

FIG. 4 shows a controller 222 of a control system 114 according to the present disclosure. The controller 222 may include one or more processors 402, memory 404 and an interface 406. The interface 406 connects (wired and/or wirelessly) the controller 222 to sensor(s), such as sensors 224, 226, 228, 230 and/or input device(s), such as input devices 204, 234, of the bioethanol system 102. The one or more processors 402 are connected to memory 404, the memory 404 storing input scheme and/or system configuration parameters or other settings relevant for the operation of controller 222.

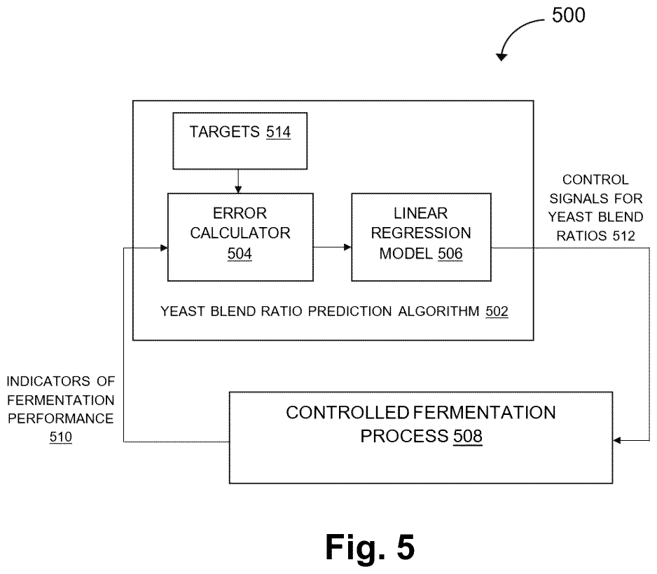

FIG. 5 illustrates an example control system 500, according to various embodiments of the present disclosure. For example only, control system 500 may include or may be part of control system 114 of FIG. 1. System 500 includes a yeast blend ratio prediction algorithm 502, which includes an error calculation unit 504 and a model 506 (e.g., a linear regression model). System 500 further includes a controlled fermentation process 508 (e.g., performed by a system such as system 102). As will be appreciated, system 500 may include one or more processors (e.g., processors 402 of FIG. 4) for carrying out various embodiments disclosed herein.

Error calculator 504 may be configured to determine an error between target data (e.g., benchmark experimental data and/or target process conditions) 514 and indicators of fermentation performance 510. For example, indicators of fermentation performance 510, which may include various indicators and/or stressors (also referred to herein as “process data”) associated with the controlled fermentation process 508. For example only, the indicators and/or stressors may include fermentation time (e.g., estimated using plant rate tags such as beer feed rate, slurry to fermentation rate, etc.), fermentation solids (e.g., estimated using solids measurements from the lab, mash density, Ronin flour mass, etc.), temperature (e.g., estimated using temperature tags from slurry, liquefaction, etc. and/or based on a temperature differential from a cooling plant that would integrate the ambient temperature outside of the plant with the ability of the facility to cool the fermentation via heat exchangers), and fermentation product and/or fermentation by-product content, such as ethanol, glycerol and organic acids (e.g., acetic acid, lactic acid, etc.) (e.g., estimated using HPLC measurements from the lab and/or or in-line measurements from one or more instruments).

Responsive to receipt of an error signal from error calculator 504, linear regression model 506 may determine, based on the error signal, a yeast blend ratio. Further, linear regression model 506 may generate one or more control signals that may be received by the controlled fermentation process 508 for controlling one or more yeast dosing skids based on the determined yeast blend ratio. Linear regression model 506 may be a least-squares regression model.

Yeast blend ratio prediction algorithm 502 may determine control signals 512 for yeast blend ratio determined by model 506. Control signals 512 may be configured to directly control actuators at controlled fermentation process 508 or may be indicative of control actions, for example only, including values for metering-in volumes of respective yeasts of a yeast blend, values for boluses of volumes of respective yeasts of a yeast blend, or values for ratios that may be utilized by a dosing control mechanism.

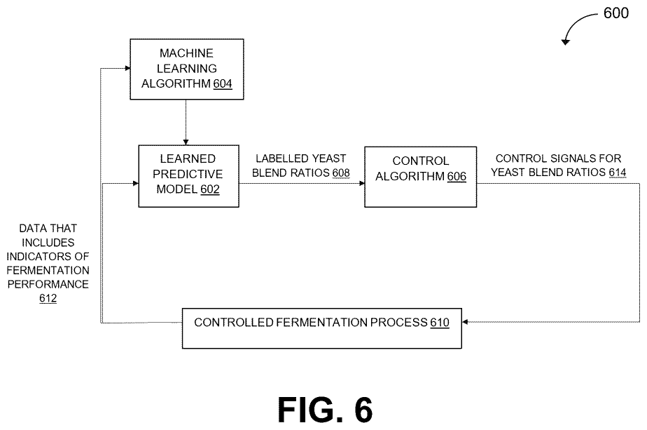

FIG. 6 illustrates an example control system 600, according to various embodiment of the present disclosure. For example only, control system 600 may include or may be part of control system 114 of FIG. 1. System 600 includes a learned predictive model 602, a machine learning algorithm 604, a control algorithm 606, and a controlled fermentation process 610 (e.g., performed by a system such as system 102). As will be appreciated, system 600 may include one or more processors (e.g., processors 402 of FIG. 4) for carrying out various embodiments disclosed herein.

The learned predictive model 602 and the machine learning algorithm 604 may be configured to receive data 612, which may include indicators of fermentation performance of controlled fermentation process 610. Such indicators of fermentation performance may be known, unknown, or a combination thereof. Similar to as described above with regard to control system 500, data 612 may include various indicators and/or stressors (also referred to herein as “process data”) associated with the controlled fermentation process 610. For example only, the indicators and/or stressors associated with the controlled fermentation process 610 may include fermentation time, fermentation solids, temperature, and fermentation products (e.g., ethanol) or fermentation by-products, such as PAN, glycerol and organic acid (e.g. acetic acid) content. Feature vectors of indicators may be continuously identified and utilized by control system 600.

Based on data 612 and/or data received from a machine learning algorithm 604, the learned predictive model 602 may label various yeast blend ratio (respectively “labelled yeast blend ratios 608”) that include information that identifies a predicted yield for a given yeast blend ratio. Control algorithm 606 may utilize the labelled yeast blend ratios 608 to determine a specific yeast blend ratio and generate one or more control signals 614 that may be received by the controlled fermentation process 610 for controlling one or more yeast dosing skids based on the determined yeast blend ratio.

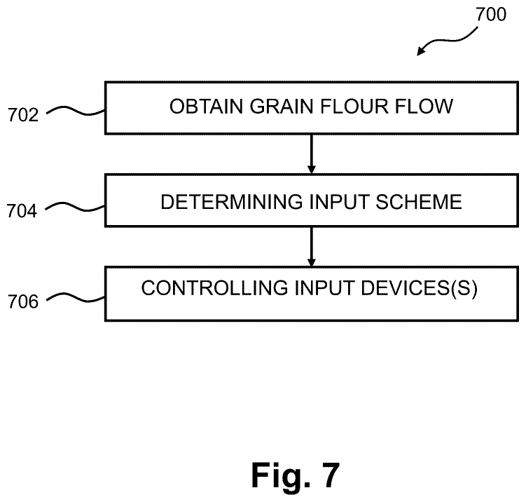

FIG. 7 shows a flowchart of an exemplary method 700 of producing bioethanol in a bioethanol system, the method 700 including obtaining a grain flour flow of grain flour in act 702, determining an input scheme based on the grain flour flow in act 704, and controlling one or more input devices of the bioethanol system according to the input scheme in act 706.

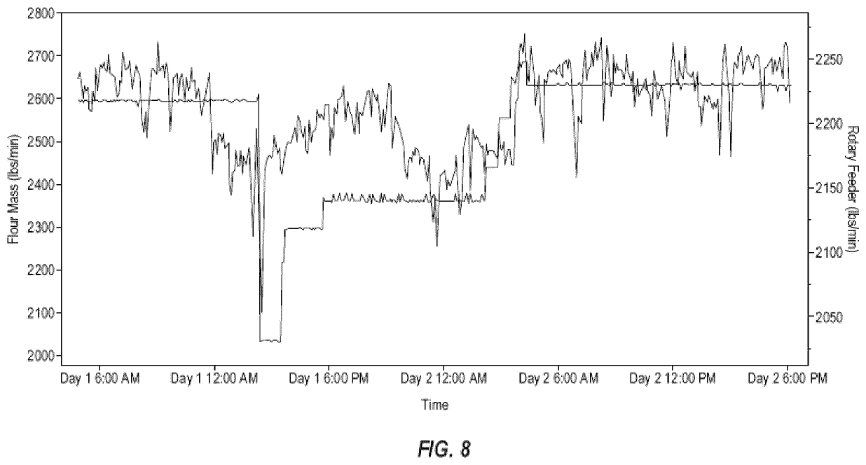

FIG. 8 shows corn mass measured in an ethanol plant by both the rotary feeder and by an embodiment of the control system of the present disclosure in which a weight meter (e.g., Ronan) is used for accurate, real-time measurement of corn mass, illustrating the error of the rotary feeder measurement, which error was predominant in the industry before the control system of the present disclosure.

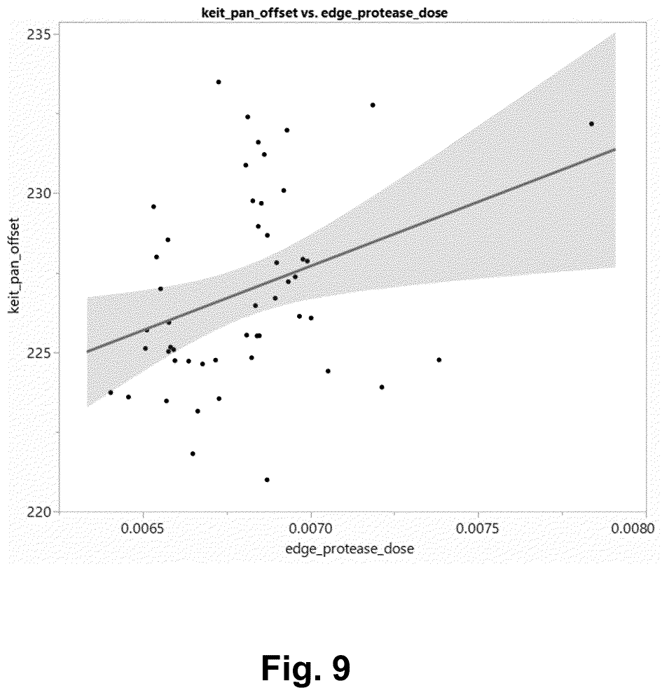

FIG. 9 illustrates the relationship between the dose of protease enzyme as a % weight of enzyme per weight of ground corn according to the weight meter and the pan_offset is the reading of primary amino nitrogen (“PAN”) from the spectrometer device. There is approximately 180 minutes from when corn is ground and enzyme added until that liquefied mash comes in contact with the spectrometer.

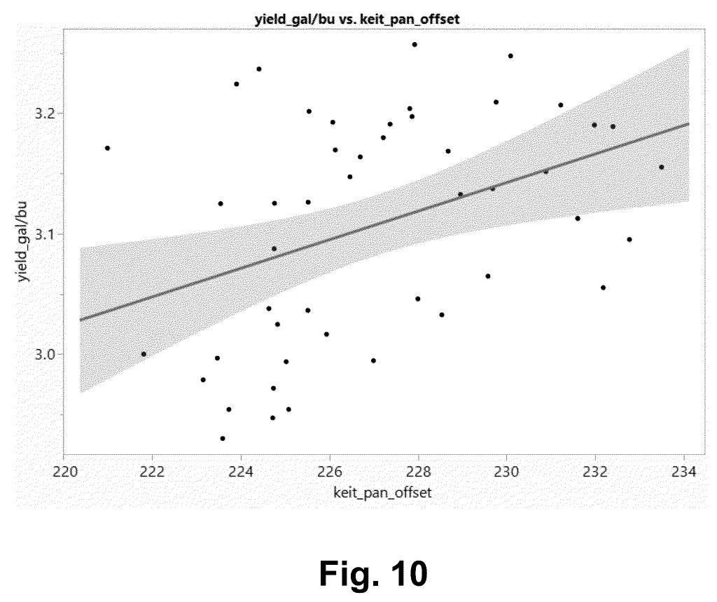

FIG. 10 illustrates the relationship between PAN reading from the spectrometer and ethanol yield. The ethanol yield of the total process is expressed in gallons of absolute ethanol per 56 pounds of corn. It reflects that increasing the PAN increases the ethanol yield.

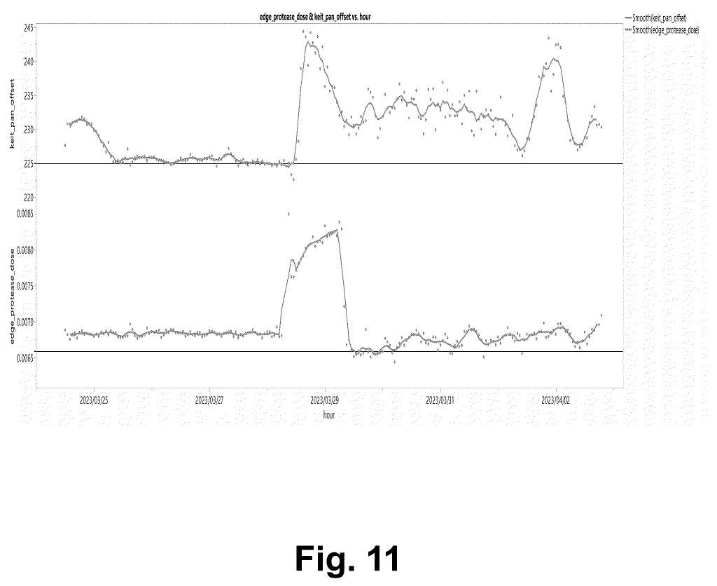

FIG. 11 illustrates the effect of protease dosing on a PAN measurement from a spectrometer device with respect to time. The red line is the protease dose as a % weight of protease enzyme per weight of corn ground. The blue line is the PAN reading from a spectrometer device. The graph highlights that the protease dose increased by −25% for 1 day and as a result, PAN stayed high for several days afterward due to the water recycling nature of the ethanol process. Excess PAN that is created initially by the protease goes through fermentation, is used by the yeast, and whatever is not used by the yeast recycles back to the cook portion of the process through backset (the water left over after distillation).

The embodiments of the disclosure described above and illustrated in the accompanying drawing figures do not limit the scope of the invention, since these embodiments are merely examples of embodiments of the invention, which is defined by the appended claims and their legal equivalents. Any equivalent embodiments are intended to be within the scope of this disclosure. Indeed, various modifications of the present disclosure, in addition to those shown and described herein, such as alternative useful combinations of the elements described, may become apparent to those skilled in the art from the description. Such modifications and embodiments are also intended to fall within the scope of the appended claims and their legal equivalents.

### EXAMPLES

### Example 1

Ethanol producers have failed to properly optimize their operations due to high process variability in the conventional dry grind ethanol production process, chiefly caused by the inability to measure their grain and thus to dose enzymes appropriately. The inability to measure grain and dose enzyme means that the mash entering fermentation is constantly changing, but those changes are largely invisible to operators. Any attempt to standardize or optimize fermentation is bound to be unsuccessful as the attempt is made against a largely invisible, moving target.

Typically, ethanol producers add enzymes into the process at a constant rate and then attempt to grind a constant amount of grain with the goal of generating a constant enzyme dose (on a weight of enzyme/weight of corn basis). The main error occurs because grain grind is controlled using the rotary feeders of the mills, which are highly inaccurate as shown in FIG. 8.

Referring to FIG. 8, when the red line shown is flat, the ethanol producers think that they are grinding a constant amount of grain, but the mass of ground flour (shown in the blue line) is actually highly variable. If the ethanol producer is pumping enzyme at a constant rate, but the grain mass is highly variable, then the resulting enzyme dose will be highly variable, as shown in the left side of FIG. 8.

The bioethanol control system of the present disclosure accurately measures flour mass and solves the above problem in two important ways. First, control logic reduces variability in grind by adjusting the rotary feeders in real time so that ethanol plant only grinds as much flour is needed to optimize their process according to predetermined targets. Second, control logic modulates the enzyme rate based on the actual amount of flour substrate so that the correct amount of enzyme is added for the amount of flour that has been ground, resulting in a stable weight/weight dose of enzyme over time. This effect is shown in the right side of FIG. 8, where the alpha-amylase (AA) rate is allowed to change with flour mass (top) resulting in a dose with much lower variability (bottom). Ultimately, this massive reduction in variability of grain flour and enzyme dose means that the mash entering each fermenter is as constant in quality as it can be. This translates into much lower variability in fermentation outcomes, which makes production more stable and predictable.

### Example 2

The control system of the present disclosure enables process control that significantly reduces observed variability in both the grinding of corn and enzyme dosing in the dry-grind ethanol process. Treating liquified biomass with a protease releases PAN that serves as growth factors for the fermenting organism, such as yeast. The release of PAN helps yeast withstand stress from high substrate and product concentrations and increasing ethanol productivity and ethanol yields. The correct measurement of the PAN levels is critical for efficient ethanol production as too little PAN content led to yeast underperformance. Existing methods to measure the PAN and adjust protease dosing are complicated and time-consuming. The bioethanol control system of the present disclosure accurately measures PAN solves the above problem.

FIG. 9 illustrates the relationship between the dose of protease enzyme as a % weight of enzyme per weight of ground corn according to the weight meter and the pan_offset is the reading of primary amino nitrogen (“PAN”) from the spectrometer device. There is approximately 180 minutes from when corn is ground and enzyme added until that liquefied mash comes in contact with the spectrometer.

FIG. 10 illustrates the relationship between PAN reading from the spectrometer and ethanol yield. The ethanol yield of the total process is expressed in gallons of absolute ethanol per 56 pounds of corn. It reflects that increasing the PAN increases the ethanol yield.

Excess PAN that is created initially by the protease goes through fermentation, is used by the yeast, and whatever is not used by the yeast recycles back to the cook portion of the process through backset (the water left over after distillation). Control logic modulates the enzyme rate based on the PAN measurement so that the correct amount of protease is added for the protein breakdown, resulting in a stable PAN stream over time. This effect is shown in FIG. 11, which illustrates the effect of protease dosing on a PAN measurement from a spectrometer device with respect to time. The red line is the protease dose as a % weight of protease enzyme per weight of corn ground. The blue line is the PAN reading from a spectrometer device. FIG. 11 highlights that the protease dose increased by −25% for 1 day and as a result, PAN stayed high for several days afterward due to the water recycling nature of the ethanol process. This translates into much lower variability in fermentation outcomes, which makes production more stable and predictable.

1-15. (canceled)

16. A control system for a bioethanol system, the control system comprising a controller comprising one or more processors and an interface, wherein the one or more processors are configured to:
obtain a primary amino nitrogen (“PAN”) measurement;
determine an input scheme based on the PAN measurement; and
control one or more input devices of the bioethanol system according to the input scheme.

17. Control system according to claim 16, wherein to determine an input scheme comprises to determine an enzyme dosing scheme, the enzyme dosing scheme comprising a first enzyme flowrate for a first enzyme, and wherein to control one or more input devices comprises to control a first enzyme input device according to the enzyme dosing scheme, wherein the first enzyme is protease.

18. Control system according to claim 17, wherein to control an input device comprises to control the first enzyme input device according to the first enzyme flowrate.

19. Control system according to claim 17, wherein the enzyme dosing scheme comprises a second enzyme flowrate for a second enzyme, and wherein to control an input device comprises to control a second enzyme input device according to the second enzyme flowrate.

20. Control system according to claim 19, wherein the second enzyme is selected from alpha-amylase, endoglucanase, glucoamylase, phospholipase, phytase, pullulanase, and xylanase or is an enzyme composition comprising one or more of alpha-amylase, endoglucanase, glucoamylase, phospholipase, phytase, pullulanase, and xylanase.

21. Control system according to claim 16, wherein to determine an input scheme comprises to determine a feed rate, and wherein to control one or more input devices comprises to control a feeder input device according to the feed rate.

22. Control system according to claim 16, wherein the one or more processes are configured to obtain a grain flour flow of grain flour.

23. Control system according to claim 16, wherein the control system comprises a weight meter for provision of weight data of grain flour and wherein to obtain a grain flour flow comprises to determine the grain flour flow based on the weight data.

24. Control system according to claim 16, wherein the control system comprises a spectrometer for provision of spectrometer data of the PAN measurement.

25. Control system according to claim 24, wherein the spectrometer is situated inside a liquefaction tank of the bioethanol system, in-line with an output of the liquefaction tank, in-line with an input of a fermentation tank of the bioethanol system, inside the fermentation tank, or in-line with an output of the fermentation tank.

26. Control system according to claim 16, wherein to determine the first enzyme flow rate for the protease enzyme is based on the PAN measurement.

27. Control system according to claim 16, wherein the control system comprises a spectrometer for provision of spectrometer data of grain flour and wherein to obtain a grain flour flow comprises to determine a first component flow of a first component of the grain flour based on the spectrometer data and the grain flour flow.

28. Control system according to claim 27, wherein the first component is protein content and wherein to obtain a grain flour flow comprises to determine a protein component flow of a protein component of the grain flour.

29. Control system according to claim 28, wherein to determine the first enzyme flow rate for the protease enzyme is based on the PAN measurement and the protein component flow.

30. Control system according to claim 28, wherein to obtain a grain flour flow comprises to determine a second component flow of a second component of the grain flour based on the spectrometer data and the grain flour flow.

31. Control system according to claim 30, wherein the second component is one of a starch, a fiber, a fat and a moisture content.

32. Method of producing bioethanol in a bioethanol system, the method comprising:
obtaining a PAN measurement;
determining an input scheme based on the PAN measurement; and
controlling one or more input devices of the bioethanol system according to the input scheme.

33. Method of claim 32, wherein the input scheme is an enzyme dosing scheme and controlling one or more input devices comprises controlling a first enzyme input device according to the enzyme dosing scheme, wherein the first enzyme is protease.

34. A control system for a bioethanol system, the control system comprising a controller comprising one or more processors and an interface, wherein the one or more processors are configured to:
obtain spectrometer data of a liquefied mash and/or of a fermented mash;
determine an input scheme based on spectrometer data of the liquefied mash and/or the fermented mash; and
control one or more input devices of the bioethanol system according to the input scheme.

35. The control system according to claim 34, wherein the spectrometer data of the liquefied mash comprises a starch flow or starch amount, a protein flow or protein amount, a fiber flow or fiber amount, and/or a fat flow and/or fat amount.

36. The control system according to claim 34, wherein the spectrometer data of the liquefied mash comprises a PAN measurement.

37. The control system according to any of claim 34, wherein the input scheme comprises a liquefaction enzyme dosing scheme, and controlling one or more input devices comprises controlling a first enzyme input device according to the enzyme dosing scheme, wherein the first enzyme is protease and a first enzyme flow rate is determined based on the protein flow or protein amount and/or the PAN measurement in the liquefied mash.

38. The control system according to claim 36, wherein the first enzyme flow rate is adjusted in real-time to achieve a target PAN measurement in the liquefied mash.

39. The control system according to claim 34, wherein the input scheme comprises a liquefaction enzyme dosing scheme, and controlling one or more input devices comprises controlling a second enzyme input device according to the enzyme dosing scheme, wherein the second enzyme is alpha-amylase and a second enzyme flow rate is determined based on the starch flow or starch amount in the liquefied mash.

40. The control system according to claim 34, wherein the input scheme comprises a liquefaction enzyme dosing scheme, and controlling one or more input devices comprises controlling a third enzyme input device according to the enzyme dosing scheme, wherein the third enzyme is xylanase and a third enzyme flow rate is determined based on the fiber flow or fiber amount in the liquefied mash.

41. The control system according to claim 34, wherein the input scheme comprises a liquefaction enzyme dosing scheme, and controlling one or more input devices comprises controlling a fourth enzyme input device according to the enzyme dosing scheme, wherein the fourth enzyme is lipase (e.g., phospholipase) and a fourth enzyme flow rate is determined based on the fat flow or fat amount in the liquefied mash.

42. The control system according to any one of claim 34, wherein the input scheme comprises a fermentation enzyme dosing scheme, and controlling one or more input devices comprises controlling a fifth enzyme input device according to the fermentation enzyme dosing scheme, wherein the fifth enzyme is alpha-amylase and a fifth enzyme flow rate is determined based on the starch flow or starch amount in the liquefied mash.

43. The control system according to claim 34, wherein the spectrometer data of the liquefied mash comprises a measurement of dextrins present in the liquefied mash.

44. The control system according to claim 34, wherein the input scheme comprises a fermentation enzyme dosing scheme, and controlling one or more input devices comprises controlling a sixth enzyme input device according to the fermentation enzyme dosing scheme, wherein the sixth enzyme is glucoamylase and a sixth enzyme flow rate is determined based on the measurement of dextrins present in the liquefied mash and/or the starch flow or starch amount in the liquefied mash.

45. The control system according to claim 34, wherein the input scheme comprises a fermentation enzyme dosing scheme, and controlling one or more input devices comprises controlling a seventh enzyme input device according to the fermentation enzyme dosing scheme, wherein the seventh enzyme is trehalase and a seventh enzyme flow rate is determined based on the measurement of trehalose present in the liquefied mash and/or the starch flow or starch amount in the liquefied mash.

46. The control system according to claim 34, wherein the input scheme comprises a fermentation enzyme dosing scheme, and controlling one or more input devices comprises controlling an eighth enzyme input device according to the fermentation enzyme dosing scheme, wherein the eighth enzyme is an enzyme composition comprising a beta-glucosidase, cellobiohydrolase, and an endoglucanase, and an eighth enzyme flow rate is determined based on the measurement of cellulose present in the liquefied mash and/or the fiber flow or fiber amount in the liquefied mash.

47. The control system according to claim 34, wherein the input scheme comprises a fermentation enzyme dosing scheme, and controlling one or more input devices comprises controlling a ninth enzyme input device according to the fermentation enzyme dosing scheme, wherein the ninth enzyme is an enzyme composition comprising an arabinofuranosidase and a xylanase, and a ninth enzyme flow rate is determined based on the measurement of hemicellulose present in the liquefied mash and/or the fiber flow or fiber amount in the liquefied mash.

48. The control system according to claim 47, wherein the ninth enzyme composition further comprises a beta-xylosidase.

49. The control system according to claim 47, wherein the ninth enzyme composition further comprises an acetyxylan esterase and/or feruloyl esterase.

50. The control system according to claim 34, wherein the input scheme comprises a fermentation enzyme dosing scheme, and controlling one or more input devices comprises controlling a tenth enzyme input device according to the fermentation enzyme dosing scheme, wherein the tenth enzyme is protease and a tenth enzyme flow rate is determined based on the measurement of PAN present in the liquefied mash and/or the protein flow or protein amount in the liquefied mash.

51. The control system according to claim 34, wherein the input scheme comprises a fermentation enzyme dosing scheme, and controlling one or more input devices comprises controlling an eleventh enzyme input device according to the fermentation enzyme dosing scheme, wherein the eleventh enzyme is phytase and an eleventh enzyme flow rate is determined based on the measurement of phytate or phytic acid present in the liquefied mash.

52. The control system according to claim 34, wherein the input scheme comprises a yeast dosing scheme, and controlling one or more input devices comprises controlling a yeast input device according to the yeast input scheme, and wherein total amount of or total flow rate of yeast is input through the yeast input device based on the PAN measurement.

53. The control system according to claim 34, wherein the spectrometer data of the fermented mash comprises a starch flow or starch amount a protein flow or protein amount, a fiber flow or fiber amount, a fat flow and/or fat amount.

54. The control system according to claim 34, wherein the spectrometer data of the fermented mash comprises a PAN measurement.

55. The control system according to claim 34, wherein the spectrometer data of the fermented mash comprises a measurement of residual starch.

56. The control system according to claim 34, wherein the input scheme comprises a fermentation enzyme dosing scheme, and controlling one or more input devices comprises controlling a fifth enzyme input device according to the fermentation enzyme dosing scheme, wherein the fifth enzyme is alpha-amylase and a fifth enzyme flow rate is determined based on the starch flow or starch amount in the fermented mash.

57. The control system according to claim 34, wherein the input scheme comprises a fermentation enzyme dosing scheme, and controlling one or more input devices comprises controlling a sixth enzyme input device according to the fermentation enzyme dosing scheme, wherein the sixth enzyme is glucoamylase and a sixth enzyme flow rate is determined based on the measurement of the starch flow or starch amount in the fermented mash.

58. The control system according to claim 34, wherein the input scheme comprises a fermentation enzyme dosing scheme, and controlling one or more input devices comprises controlling a seventh enzyme input device according to the fermentation enzyme dosing scheme, wherein the seventh enzyme is trehalase and a seventh enzyme flow rate is determined based on the measurement of trehalose present in the fermented mash.

59. The control system according to claim 34, wherein the input scheme comprises a fermentation enzyme dosing scheme, and controlling one or more input devices comprises controlling an eighth enzyme input device according to the fermentation enzyme dosing scheme, wherein the eighth enzyme is an enzyme composition comprising a beta-glucosidase, cellobiohydrolase, and an endoglucanase, and an eighth enzyme flow rate is determined based on the measurement of cellulose present in the fermented mash and/or the fiber flow or fiber amount in the fermented mash.

60. The control system according to claim 34, wherein the input scheme comprises a fermentation enzyme dosing scheme, and controlling one or more input devices comprises controlling a ninth enzyme input device according to the fermentation enzyme dosing scheme, wherein the ninth enzyme is an enzyme composition comprising an arabinofuranosidase and a xylanase, and a ninth enzyme flow rate is determined based on the measurement of hemicellulose present in the fermented mash and/or the fiber flow or fiber amount in the fermented mash.

61. The control system according to claim 34, wherein the eighth enzyme composition further comprises a beta-xylosidase.

62. The control system according to claim 60, wherein the eighth enzyme composition further comprises an acetyxylan esterase and/or feruloyl esterase.

63. The control system according to claim 34, wherein the input scheme comprises a fermentation enzyme dosing scheme, and controlling one or more input devices comprises controlling a tenth enzyme input device according to the fermentation enzyme dosing scheme, wherein the tenth enzyme is protease and a tenth enzyme flow rate is determined based on the measurement of PAN present in the fermented mash and/or the protein flow or protein amount in the fermented mash.

64. The control system according to claim 34, wherein the input scheme comprises a fermentation enzyme dosing scheme, and controlling one or more input devices comprises controlling an eleventh enzyme input device according to the fermentation enzyme dosing scheme, wherein the eleventh enzyme is phytase and an eleventh enzyme flow rate is determined based on the measurement of phytate or phytic acid present in the fermented mash.
